(19) United States
(12) Patent Application Publication
(10) Pub. No.: US 2025/0163099 A1
(43) Pub. Date: May 22, 2025

(54) ANISOTROPIC LIGHT-EMITTING COLLOIDAL KAGOME SUPERLATTICES

(71) Applicant: NORTHWESTERN UNIVERSITY, Evanston, IL (US)

(72) Inventors: Chad A. Mirkin, Wilmette, IL (US); Zhiwei Li, Evanston, IL (US); Wenjie Zhou, Evanston, IL (US)

(21) Appl. No.: 18/957,093

(22) Filed: Nov. 22, 2024

Related U.S. Application Data
(60) Provisional application No. 63/601,889, filed on Nov. 22, 2023.

(51) Int. Cl. C07H 23/00; B01D 9/00; B82Y 40/00

(52) U.S. Cl. CPC C07H 23/00; B01D 9/0013; B01D 9/0036; B82Y 40/00; B01D 2009/0086

(57) ABSTRACT

A method of making colloidal kagome superlattices can include functionalizing a plurality of metal nanoparticles with a plurality of oligonucleotides to produce programmable atom equivalents; and cooling the programmable atom equivalents to induce crystallization of the programmable atom equivalents. The cooling process can include cooling the PAEs from a first temperature to a second temperature at a first cooling rate, cooling the PAEs from the second temperature to a third temperature at a second cooling rate, and cooling the PAEs from the third temperature to a fourth temperature at a third cooling rate. The kagome superlattices can have a rhombohedral unit cell formed by alternating chiral layers of PAEs organized in 2-dimensional distorted kagome layers. The colloidal superlattices can have anisotropic light emission, Purcell factors of at least 25 and birefringent properties.

### CROSS REFERENCE TO RELATED APPLICATION

The benefit of priority to U.S. Provisional Application No. 63/601,889 filed Nov. 22, 2023, is hereby claimed and the disclosure is incorporated herein by reference in its entirety.

### STATEMENT OF GOVERNMENT SUPPORT

This invention was made with government support under grant numbers FA9550-22-1-0300 and FA9550-17-1-0348 awarded by the Air Force Office of Scientific Research. The government has certain rights in the invention.

### INCORPORATION BY REFERENCE OF THE SEQUENCE LISTING

This application contains, as a separate part of disclosure, a Sequence Listing in computer-readable form (filename: 59677_SeqListing.xml; Size: 4,416 bytes: Created: Nov. 21, 2024) which is incorporated by reference herein in its entirety.

### FIELD

The disclosure is generally directed to methods of assembling colloidal superlattices with anisotropic light-emitting properties.

### BACKGROUND

Understanding the assembly of atomic and nanoscale building blocks into crystals is one of the cornerstones of chemistry and materials science as it is central to creating functional materials with properties by design. It is well established that targeting a set of defined properties depends on both the composition of a material and its structure, where crystal parameters such as spacing, orientation, and habit are of particular interest. Routes to deliberately tune atomic, molecular and nanoscale ordering across length scales have led to a swath of important materials with exotic photonic and mechanical properties. For example, control over periodicity and symmetry of superlattices enables the design of colloidal crystals with tunable optical diffraction, super-fluorescence, lattice plasmon resonance, and negative refraction, which are not attainable in individual nanocrystals. As synthetic methods for producing nanoparticles of different compositions, sizes and crystallinity are developed, colloidal assembly provides access to new materials with exotic properties by design.

DNA-mediated programmable assembly of nanomaterials has emerged as one of the most powerful and versatile ways to control colloidal crystal structures (composition, symmetry, lattice parameter, and habit). In colloidal crystal engineering with DNA, collections of base-pairs act as physical bonds that are decoupled from the identity of the nanoparticle cores or “atoms”. Through well-established routes, nanoparticle cores of diverse shapes, compositions and sizes can be modified with deliberately designed DNA strands to create ‘programmable atom equivalents’ (PAEs). The assembly of PAEs into colloidal crystal structures follows the complementary contact model (CCM), which operates under the premise that the thermodynamically stable structure will be the one maximizing complementary DNA interactions between building blocks. This model has been used to design and assemble crystals spanning over 90 different symmetries to date.

### SUMMARY

In accordance with methods of the disclosure, low-symmetry colloidal kagome superlattices can be synthesized from DNA-modified metal bipyramids that can engage in partial DNA surface matching. The particle dimensions and DNA length can be tuned to assemble the bipyramids into colloidal crystals with rhombohedral unit cells, including one comprised of a periodic stacking of kagome layers. Enabled by the partial facet alignment, the kagome layers exhibit lattice distortion, bipyramid twisting and planar chirality. Advantageously, such low-symmetry structures feature complex lattice plasmon resonance modes dependent on the light incident direction and polarization, making these crystals applicable for novel lasers, displays, and optical sensing constructs. When conjugated with Cy-5 molecular dyes, these kagome colloidal crystals can serve as cavities with high-density optical states and can have large Purcell factors along two lateral directions, leading to strong dipole radiation along the z-axis and facet-dependent, anisotropic light emission.

A method of making colloidal kagome superlattices using programmable atom equivalents (PAEs) in accordance with the disclosure can include preparing programmable atom equivalents by providing a solution containing pentagonal nanoparticle seeds, admixing in solution the nanoparticle seeds with one or more metal ions, and a surfactant, ageing the solution to grow surfactant stabilized metal nanoparticles each having a bipyramid shape and surfactant molecules weakly bounded to their surface. Preparing the PAEs further includes admixing the surfactant stabilized metal nanoparticles with oligonucleotides, each including an anchor region and a linker region with a self-complementary GCGC sticky end to functionalize the metal nanoparticles with the oligonucleotides. The method further includes cooling the PAEs to induce crystallization, the PAEs are adapted to arrange in a desired crystalline structure through hybridization of sticky ends of oligonucleotides of one PAE with a sticky ends of oligonucleotides of another PAE. Cooling can be performed in three stages, for example, cooling the PAEs from a first temperature to a second temperature at a first cooling rate, then from the second temperature to a third temperature at a second cooling rate, and then from the third temperature to a fourth temperature at a third cooling rate. The first temperature is higher than the second temperature, the second temperature is higher than the third temperature, and the fourth temperature is about room temperature.

A colloidal kagome superlattice of programmable PAEs according to the disclosure can include a rhombohedral unit cell formed by 3-right handed and 3-left handed chiral layers of PAEs. The PAEs can be pentagonal metal bipyramids with oligonucleotides attached to an outer surface, the layers stack in alternating chirality, i.e., each right-handed layer can be stacked between two left-handed layers, each layer can include a plurality of bipyramids organized in trimers that upon hybridization of the oligonucleotides twist to form a 2D distorted kagome pattern characterized by tiling of equilateral triangles and hexagonal shields.

### BRIEF DESCRIPTION OF THE DRAWINGS

FIG. 1A is a dark field scanning transmission electron microscopy (STEM) image of Au bipyramids.

FIG. 1B is a schematic illustration of the DNA-functionalized Au bipyramids.

FIG. 1C-1G are a series of SEM image of colloidal crystals showing (C) a collection of kagome superlattice crystals, (D) the top {111}facets of kagome superlattices, (E) a side-view of the bipyramid assembly, (F) {100}facets of the rhombohedral superlattices and (G) {110}facets of the rhombohedral superlattices.

FIG. 1H is a cross-sectional TEM image of bipyramids assembled into kagome superlattices.

FIG. 11 is a schematic illustration of a single kagome superlattice and its stacking within the superlattice.

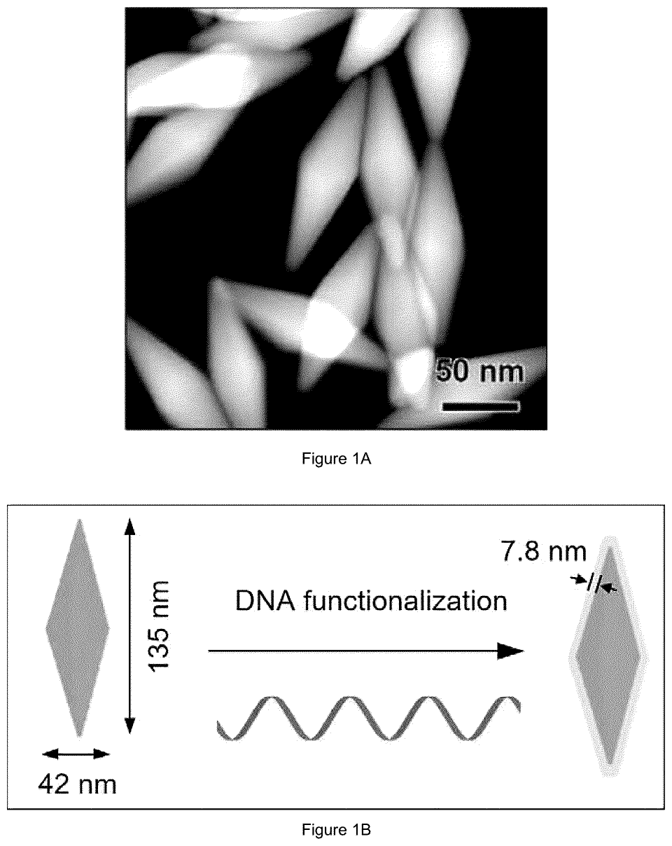

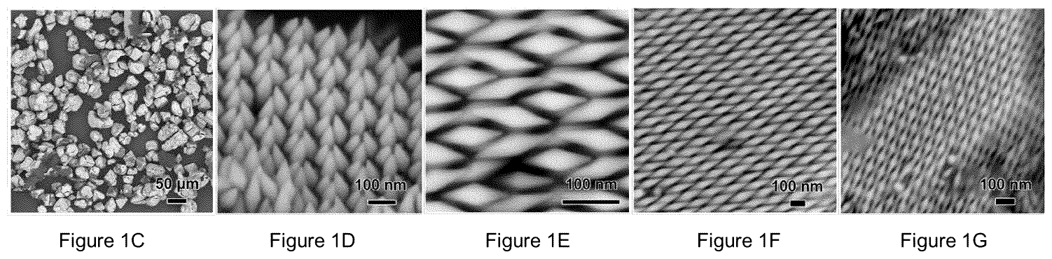

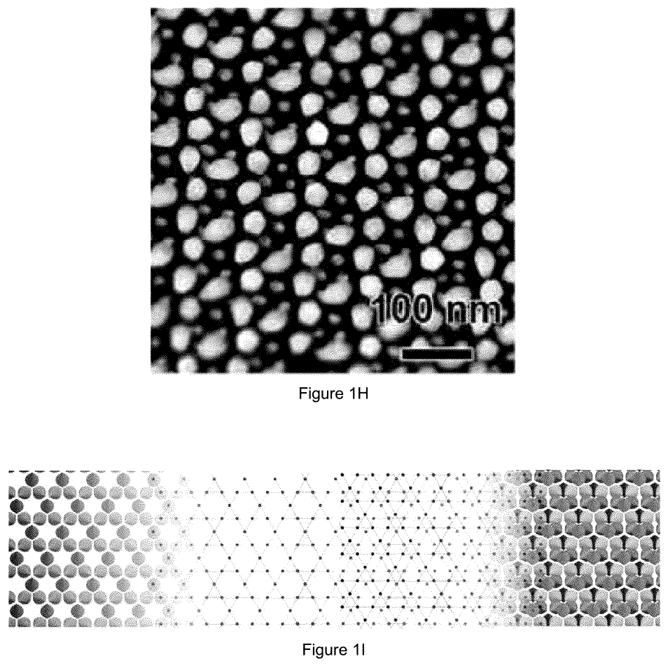

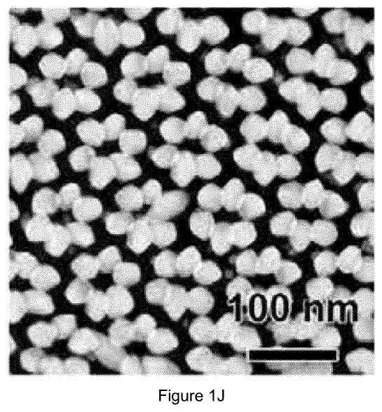

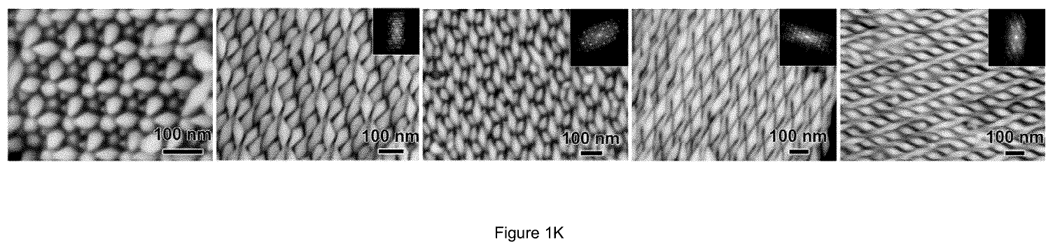

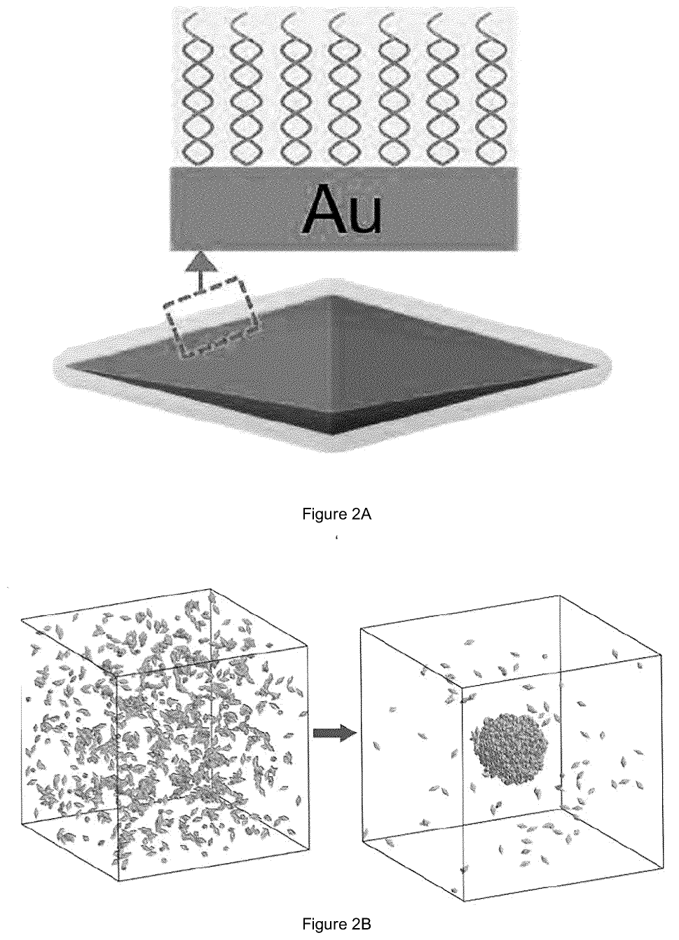

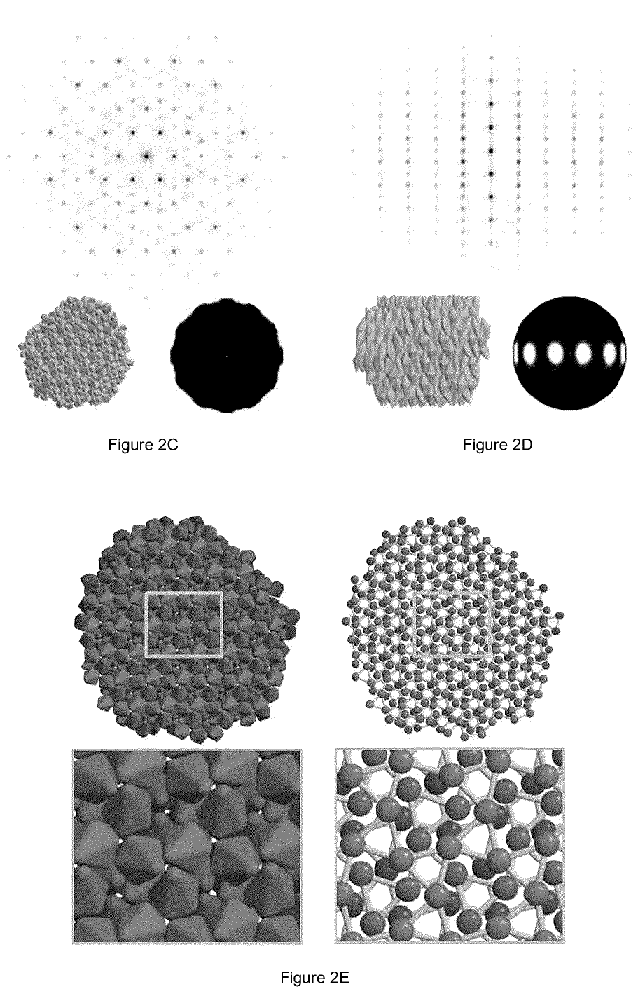

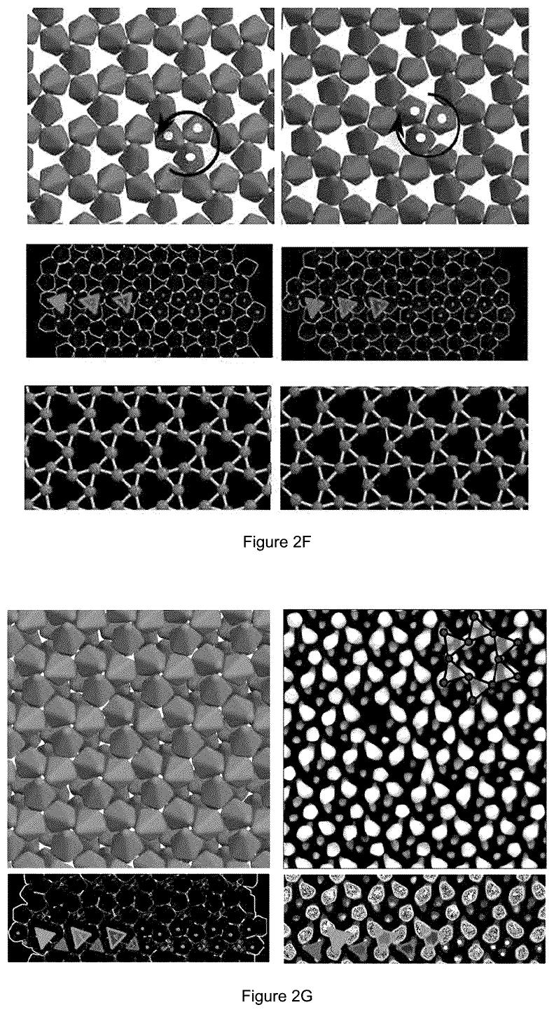

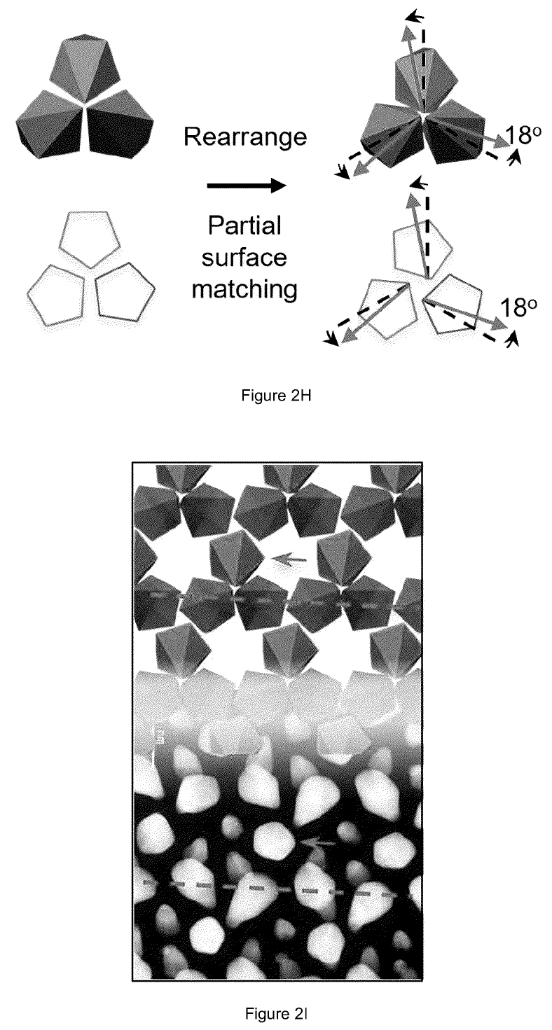

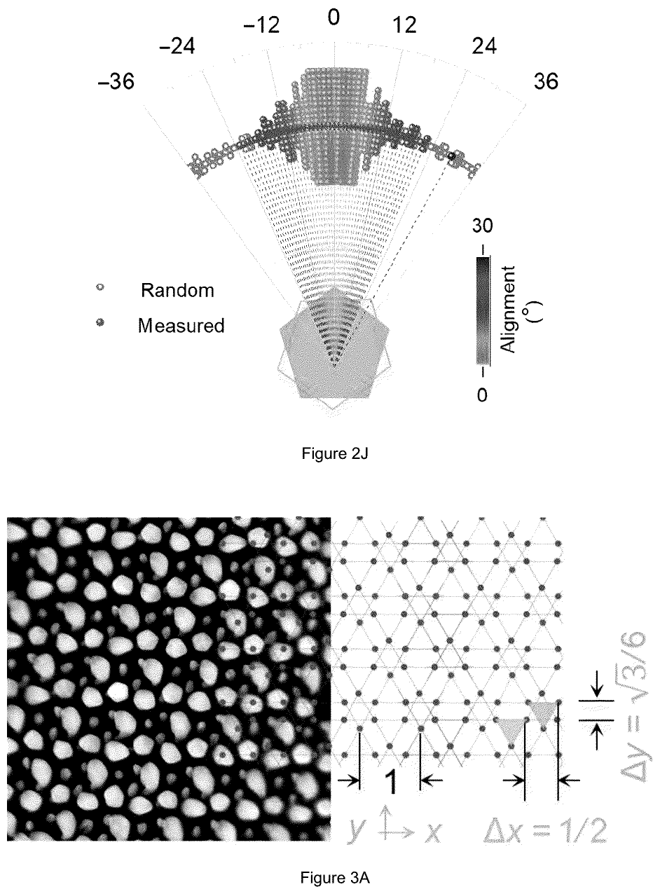

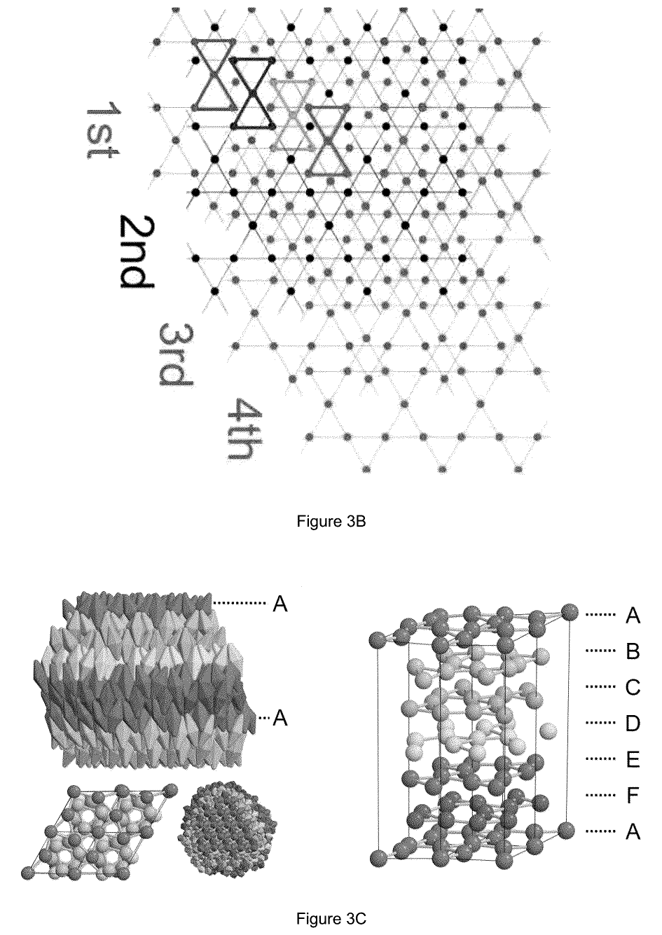

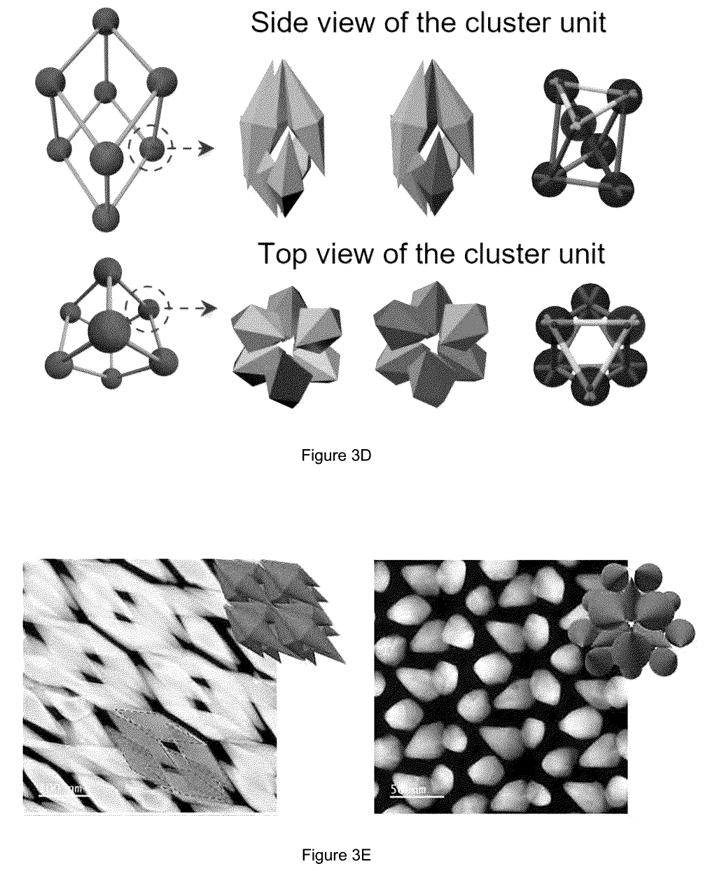

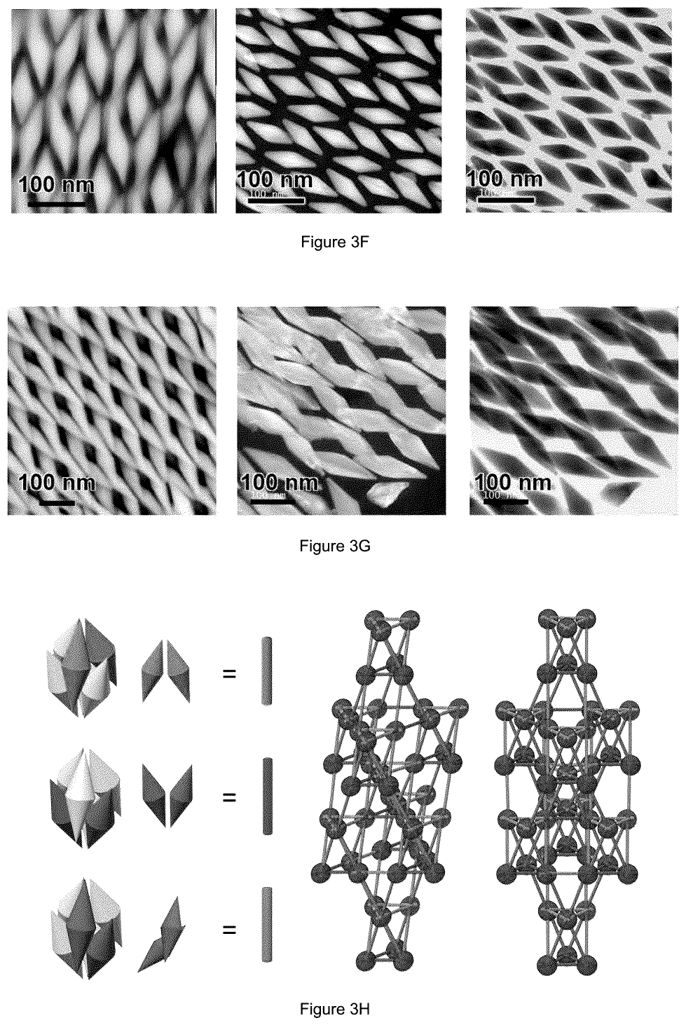

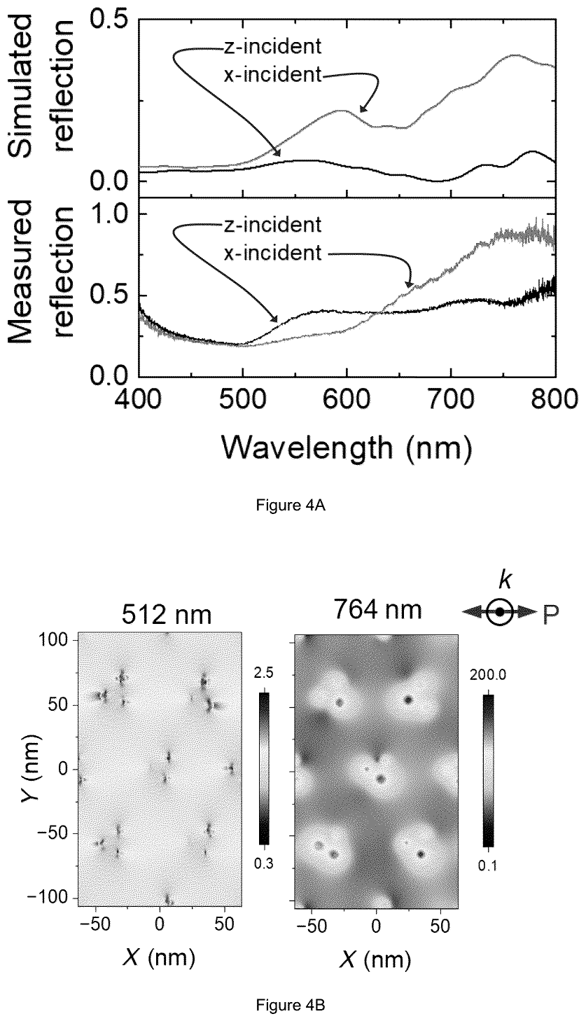

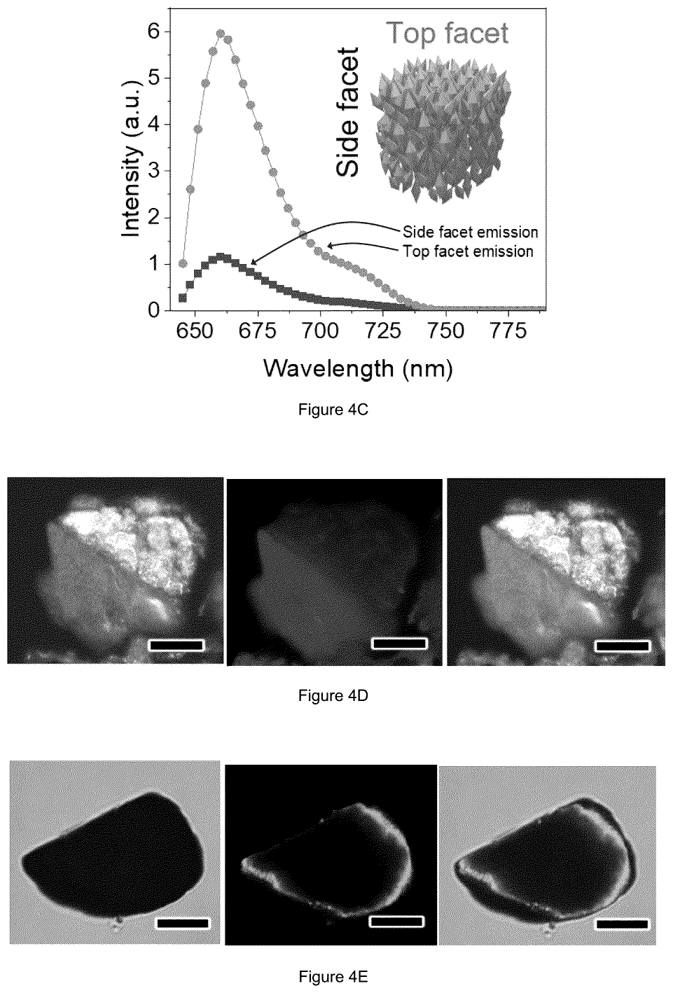

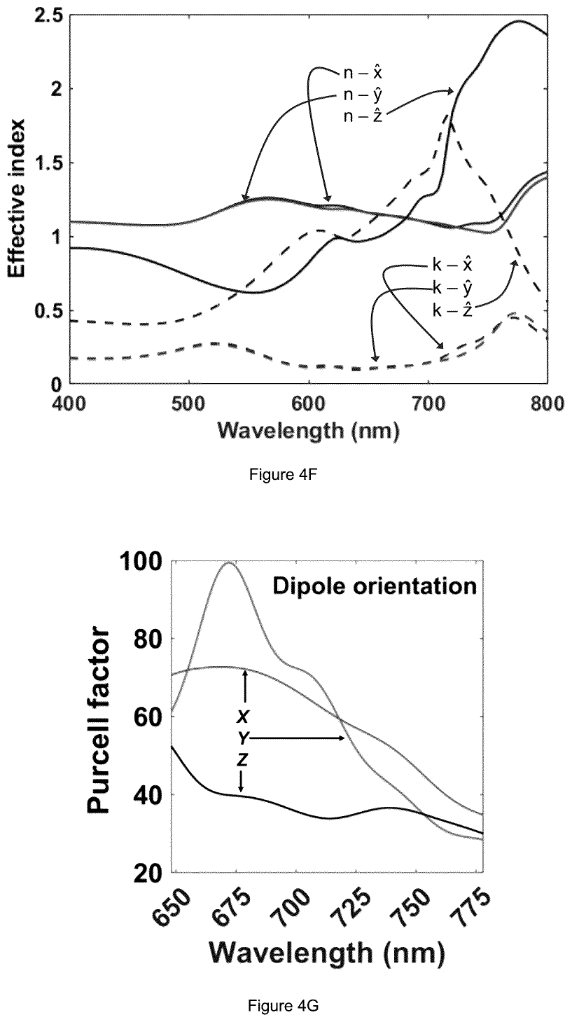

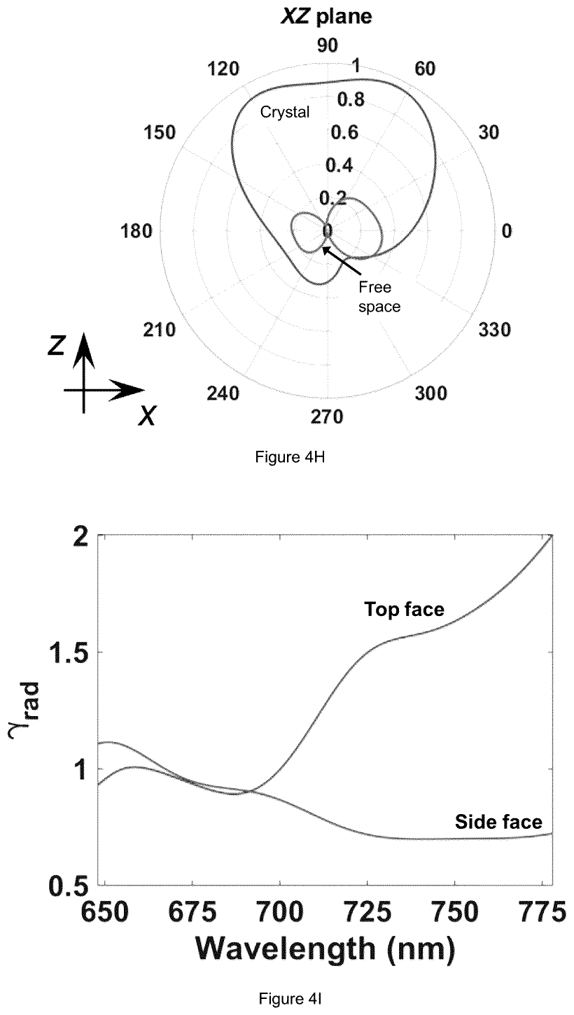

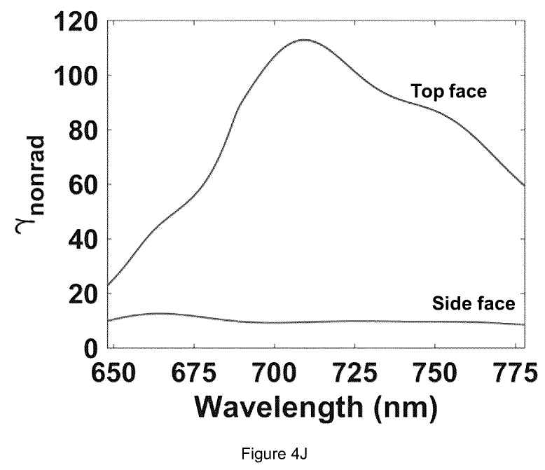

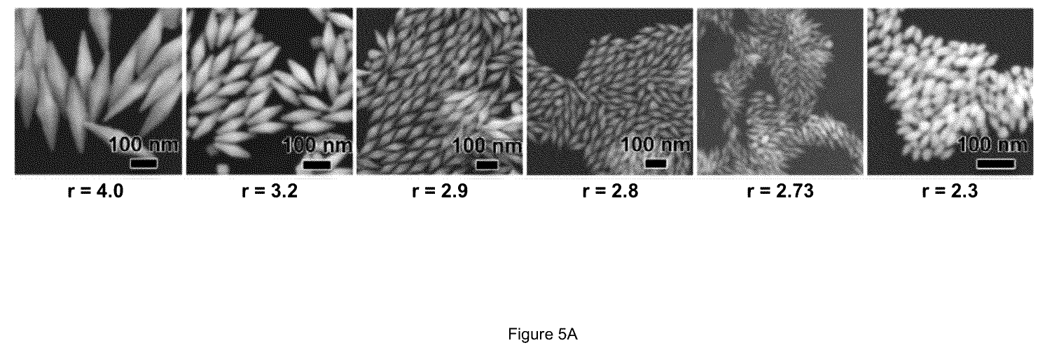

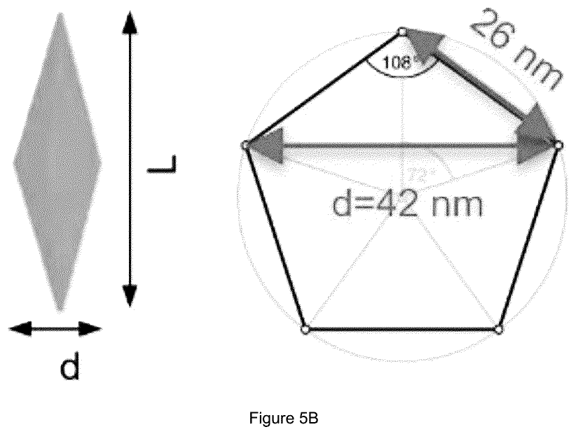

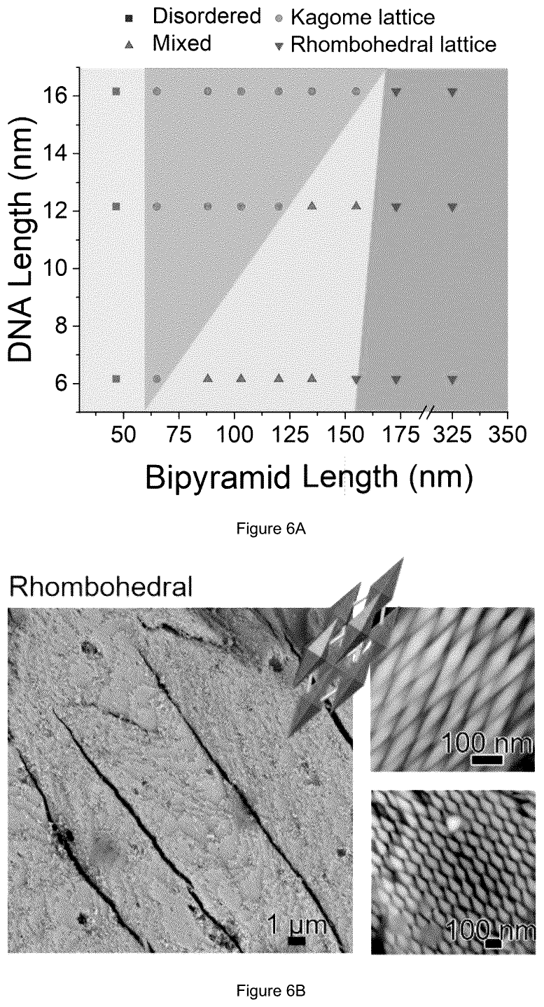

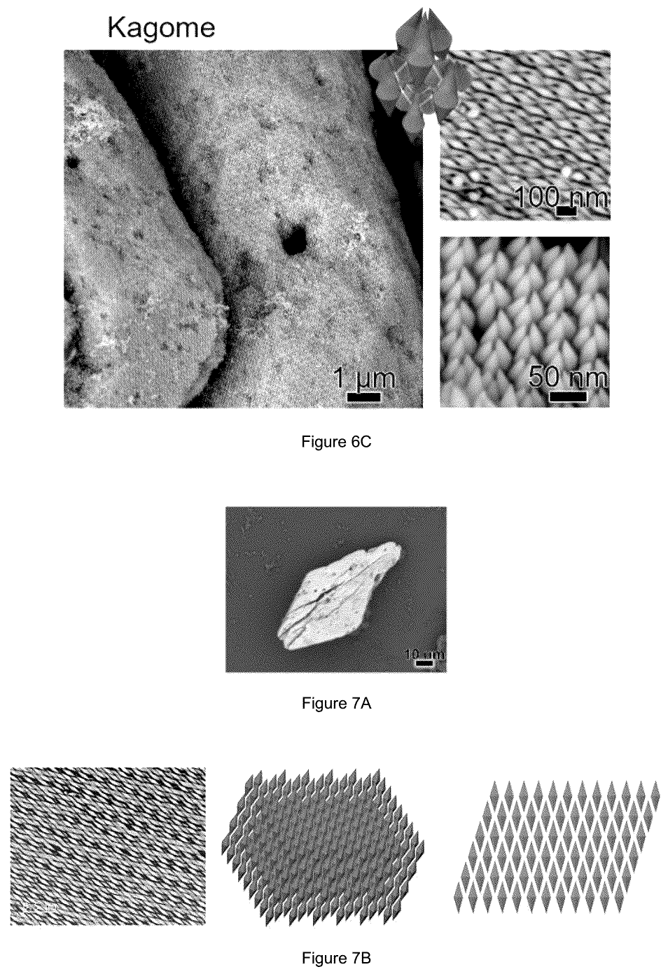

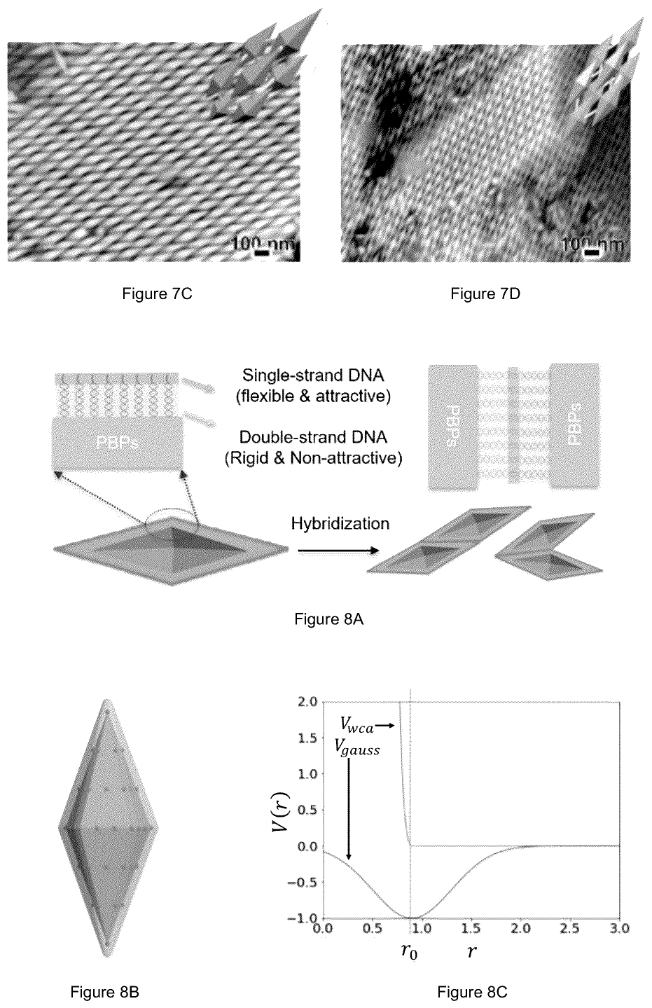

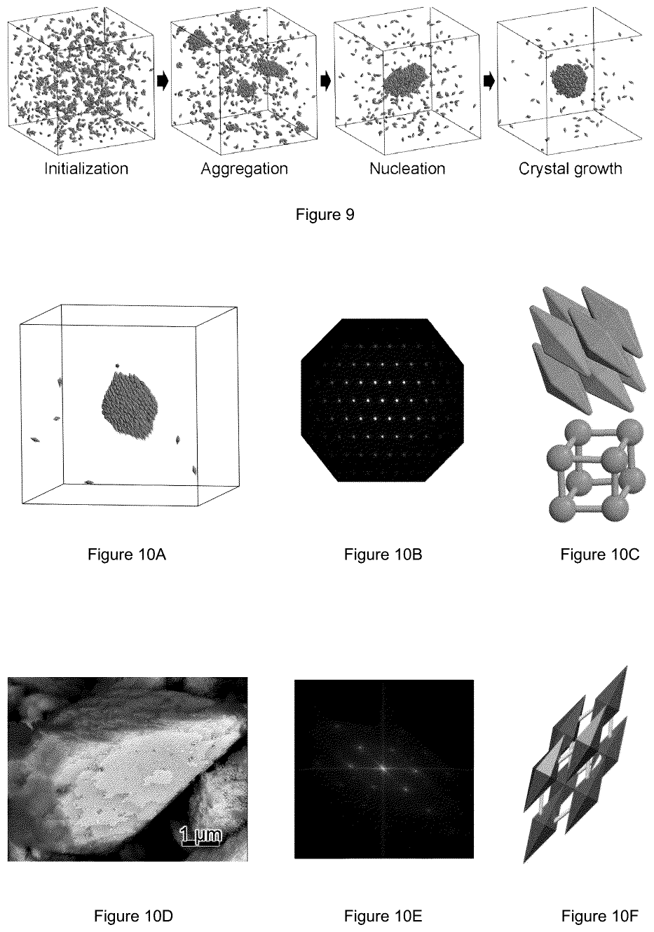

FIG. 1J is a cross-sectional TEM image containing two adjacent kagome superlattices.

FIG. 1K is a series of SEM images of the diverse assembly motifs of bipyramids in the kagome superlattices.

FIG. 2A is a schematic illustration of a collection of DNA strands on the surface of a Au bipyramid.

FIG. 2B is schematic of the molecular dynamic simulation showing the assembly of randomly dispersed Au bipyramids into a superlattice.

FIGS. 2C and 2D are FFT patterns of an assembled crystal calculated from different orientations: along (C) and perpendicular (D) to the kagome lattice stacking direction. The insets are snapshots of the crystal (bottom left) and bond-orientational order diagram (bottom right).

FIG. 2E shows two views of the stacking of three layers stacked in a kagome superlattice and corresponding zoomed-in views on the bottom. Each sphere represents the center of a bipyramid and the bonds connecting the nearest neighbors show the tiling with equilateral triangles and shields.

FIG. 2F shows local bipyramid motifs in two adjacent kagome layers the bipyramid twisting (black arrows), facet alignment, and superlattice distortion are shown in the top, middle, and bottom panels, respectively.

FIG. 2G is a schematic illustration and a cross-sectional TEM image of the relative lattice and lattice distortion in two adjacent kagome layers.

FIG. 2H is a schematic illustration of the bipyramids twisting through partial facet alignment.

FIG. 2I is a schematic illustration and a cross-sectional TEM image of the alignment of one of pentagonal domain boundaries to the superlattice orientation within the kagome layers.

FIG. 2J is a distribution of the angles between one of the domain boundaries and one lattice orientation in the distorted kagome superlattice.

FIG. 3A is a cross-sectional TEM image of a two-layer kagome superlattice and relative lattice position offset.

FIG. 3B is a schematic stacking of four kagome layers into 3D superlattices.

FIG. 3C is a snapshot of the simulated 3D superlattice with multilayer stacking. The first and the seventh layers (marked A) are identical, demonstrating a six-layer repeat pattern.

FIG. 3D is a schematic illustration of the rhombohedral unit cell and the registration of a six-bipyramid cluster at each lattice point.

FIG. 3E is pair of high-angle annular dark-field (HAADF)-STEM images of the side (top image) and top (bottom image) views of a six-bipyramid cluster.

FIG. 3F are SEM (left), HAADF-STEM (middle), and bright field-STEM (right) images of the bipyramids in parallel bonding.

FIG. 3G are SEM (left), HAADF-STEM (middle), and bright field-STEM (right) images of the bipyramids in staggered bonding.

FIG. 3H are ball- and stick models of the bonding states of bipyramids in a rhombohedral unit cell.

FIG. 4A Measured and simulated reflection spectra of one single crystal under different light incident directions.

FIG. 4B are heatmaps of the simulation results of the electric field mapping in the x-y plane under light excitation at 512 and 764 nm.

FIG. 4C are fluorescence spectra measured on different facets of the superlattices.

FIG. 4D are optical microscopy (left), fluorescence (middle), and merged images (right) of a single crystal according to the disclosure.

FIG. 4E are bright-field (left), fluorescence (middle), and merged confocal images (right) of a single crystal according to the disclosure.

FIG. 4F are simulated effective refractive indices of the kagome superlattices along different directions.

FIG. 4G are simulated Purcell factor curves of a dipole located inside the crystal and directed along the x, y, and z axes.

FIG. 4H are simulated radiation patterns over the xz plane of a random dipole cloud located inside the crystal and in the free space.

FIG. 4I are simulated average radiative decay rates of dipoles located at the top and side faces of the crystal.

FIG. 4J are simulated average nonradiative decay rates of dipoles located at the top and side faces of the crystal.

FIG. 5A is a series of SEM images of Au bipyramids of different aspect ratios.

FIG. 5B is a schematic illustration of the bipyramid dimensions.

FIG. 6A is a phase diagram of the assembled superlattices of bipyramids of different lengths and DNA strands of various lengths.

FIG. 6B are SEM images of the rhombohedral lattices featuring unidirectional alignment of bipyramids.

FIG. 6C are SEM images of the kagome superlattices containing bipyramids with three different orientations.

FIG. 7A is an SEM image of a single rhombohedral crystal.

FIG. 7B is a cross-sectional TEM image of a rhombohedral crystal.

FIG. 7C is an SEM image (top panel) and a schematic illustration (bottom panel) of one facet of a rhombohedral crystal.

FIG. 7D is an SEM image (top panel) and a schematic illustration (bottom panel) of a different facet of a rhombohedral crystal.

FIG. 8A is a schematic illustration of the assembly pathway and the stacking of the kagome lattices into colloidal crystals.

FIG. 8B is a schematic illustration of the attractive patches on the bipyramid surface.

FIG. 8C is a plot of the purely repulsive WCA potential that models hard core and rigid shell and the attractive shifted-Gaussian potential that models DNA-hybridization.

FIG. 9 is a series of simulation snapshots of the self-assembly of kagome superlattices taken at 0, 1.3×10⁶, 4.3×10⁶, 32.5×10⁶ MD time steps. The crystal phase is shown in the center of the last frame.

FIG. 10A-10C show molecular dynamics simulation results of formation of a rhombohedral superlattice. (A) is a schematic of the formation of the superlattice (bipyramid aspect ratio=2.6, r0=0.5), (B) is the FFT, and (C) is a schematic of the unit cell of the self-assembled rhombohedral superlattice.

FIG. 10D-1 OF show experimental observations of the formation of a rhombohedral superlattice. (D) is an SEM image of the crystal, (E) is the FFT, and (F) is a schematic unit cell of the rhombohedral colloidal crystals.

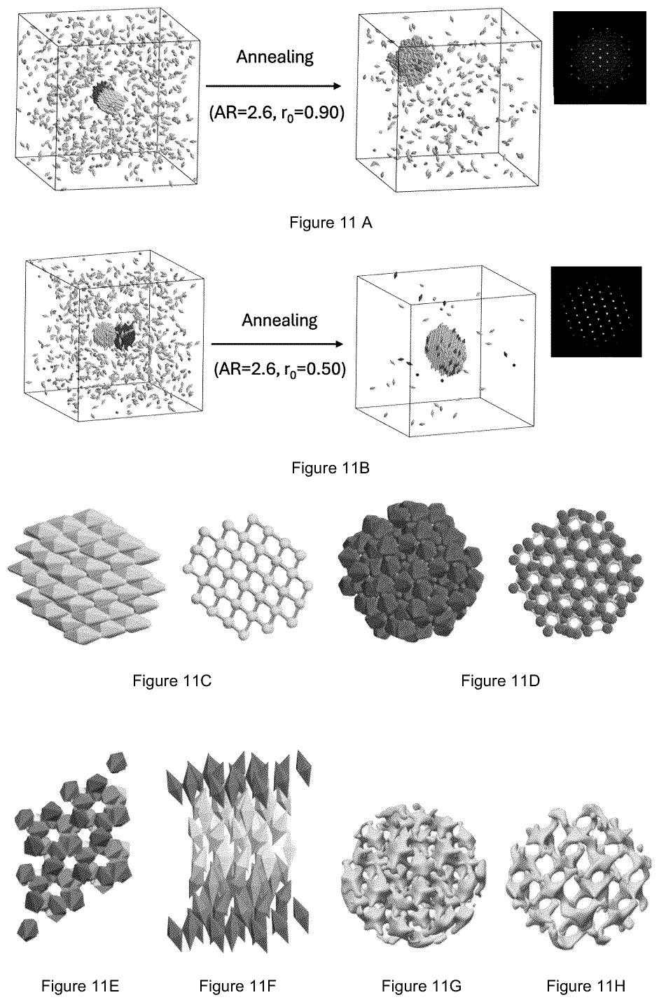

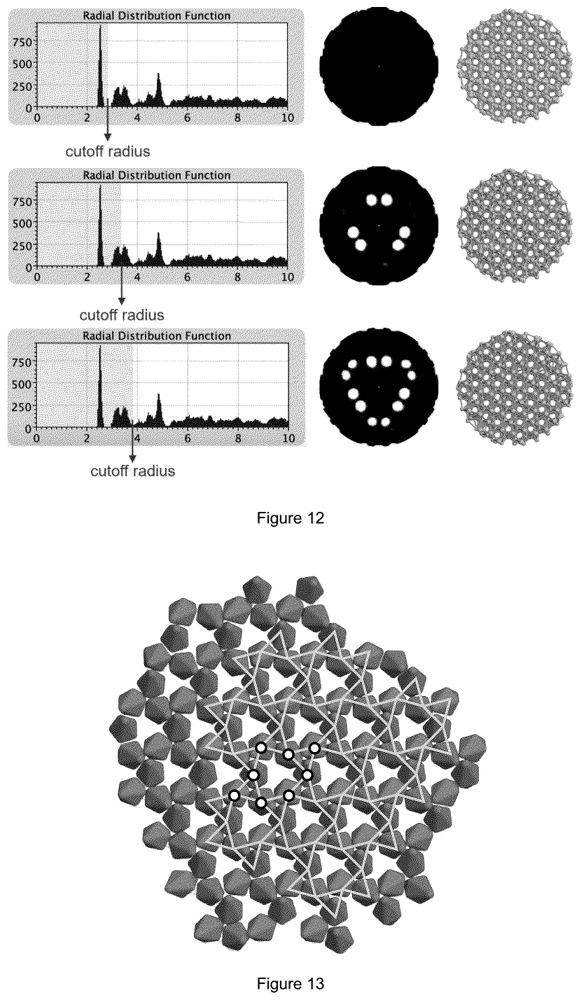

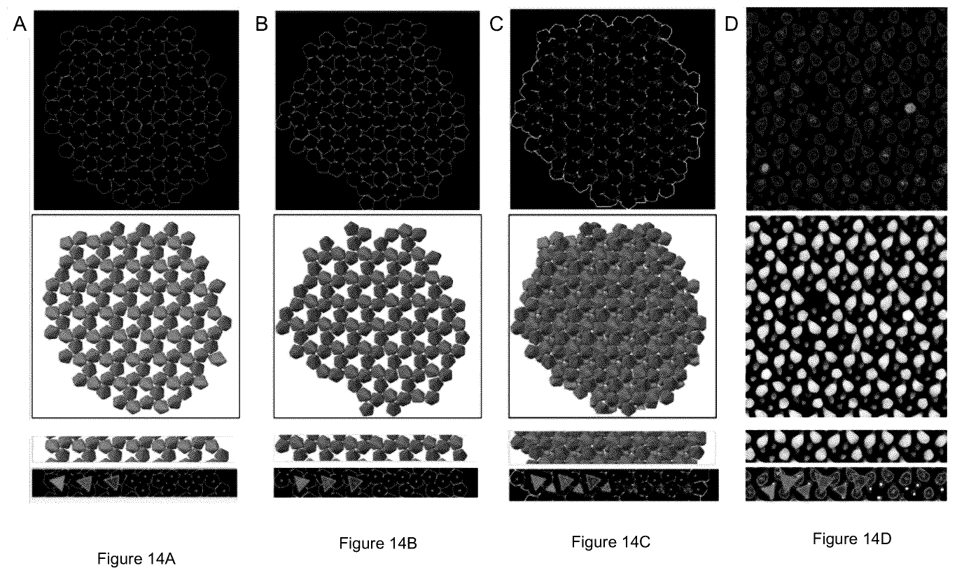

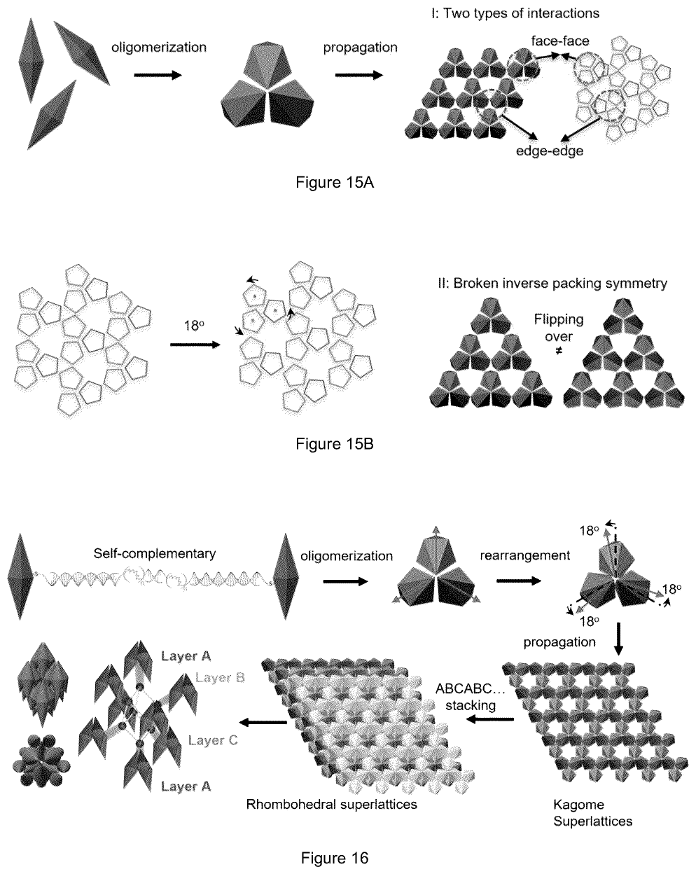

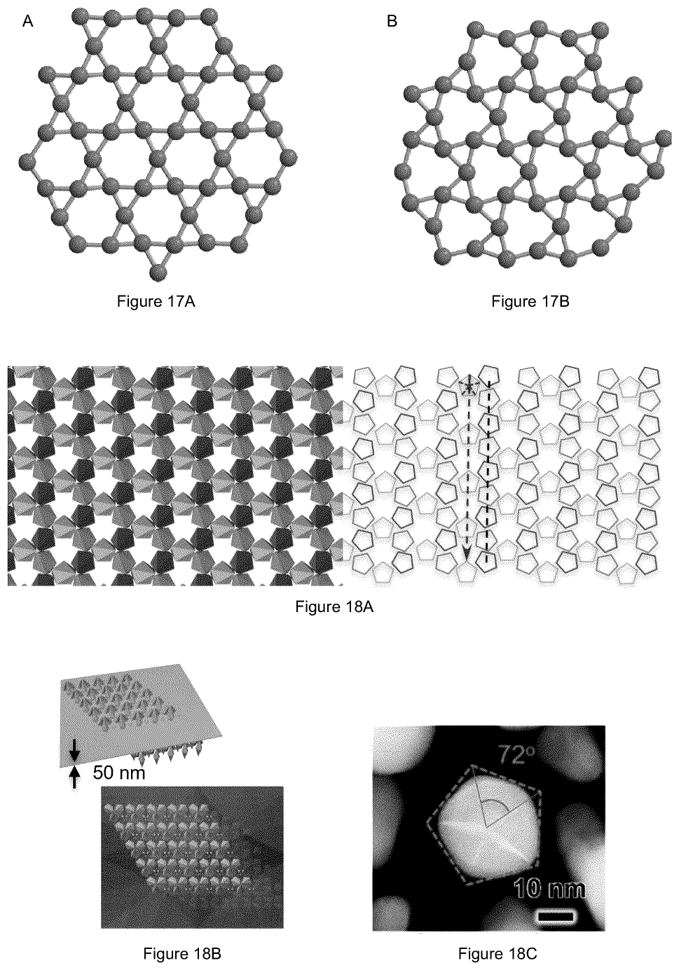

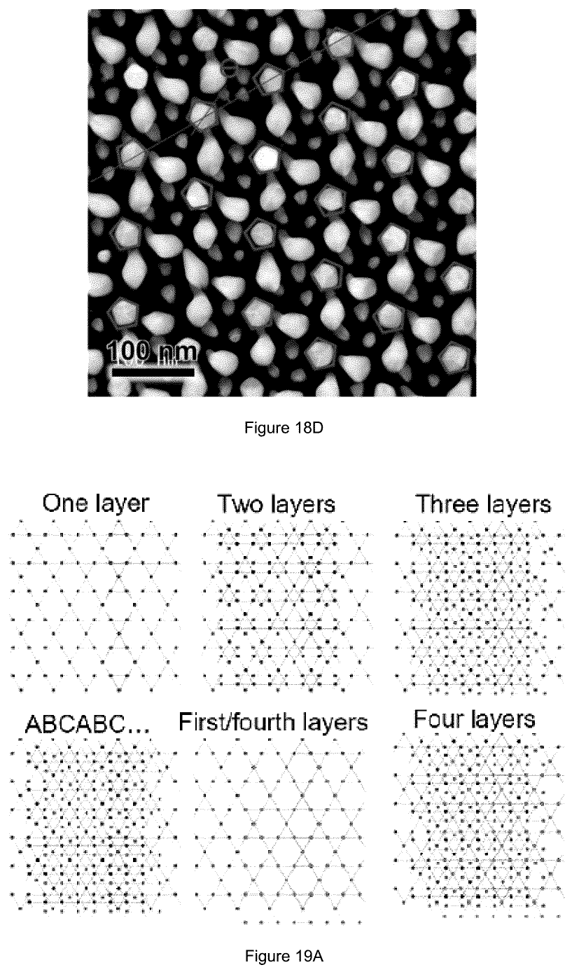

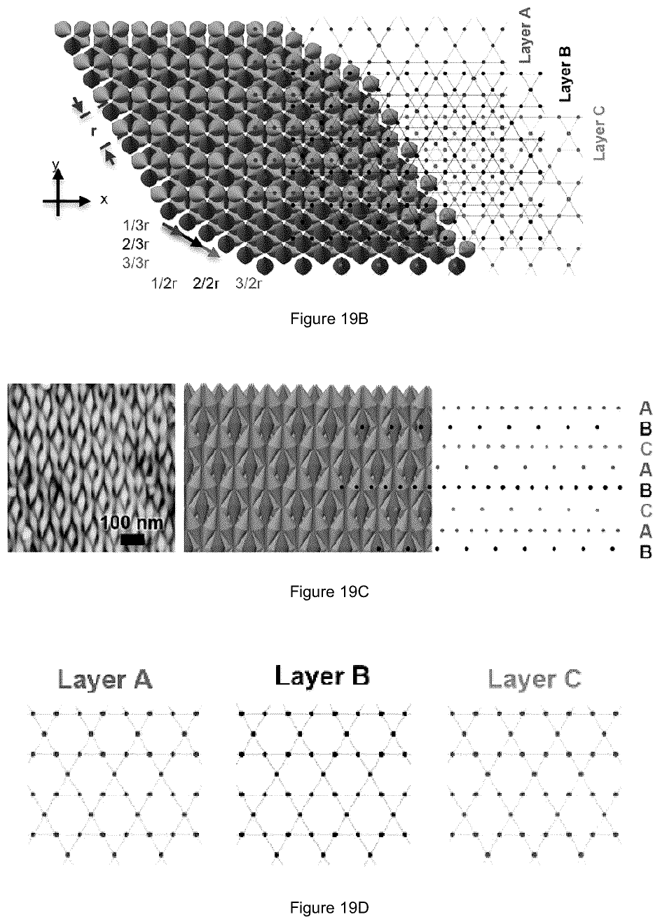

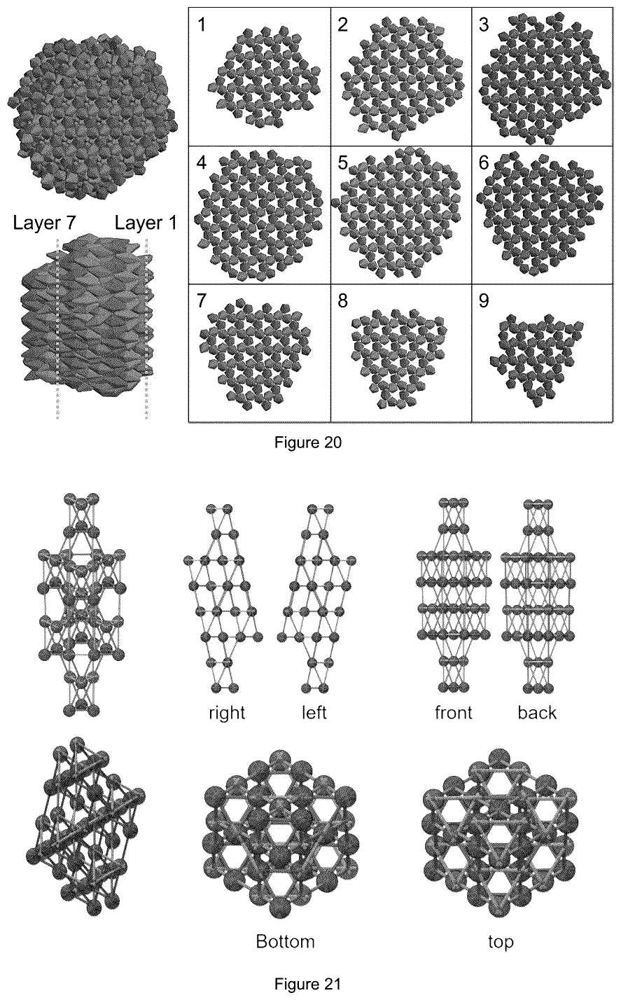

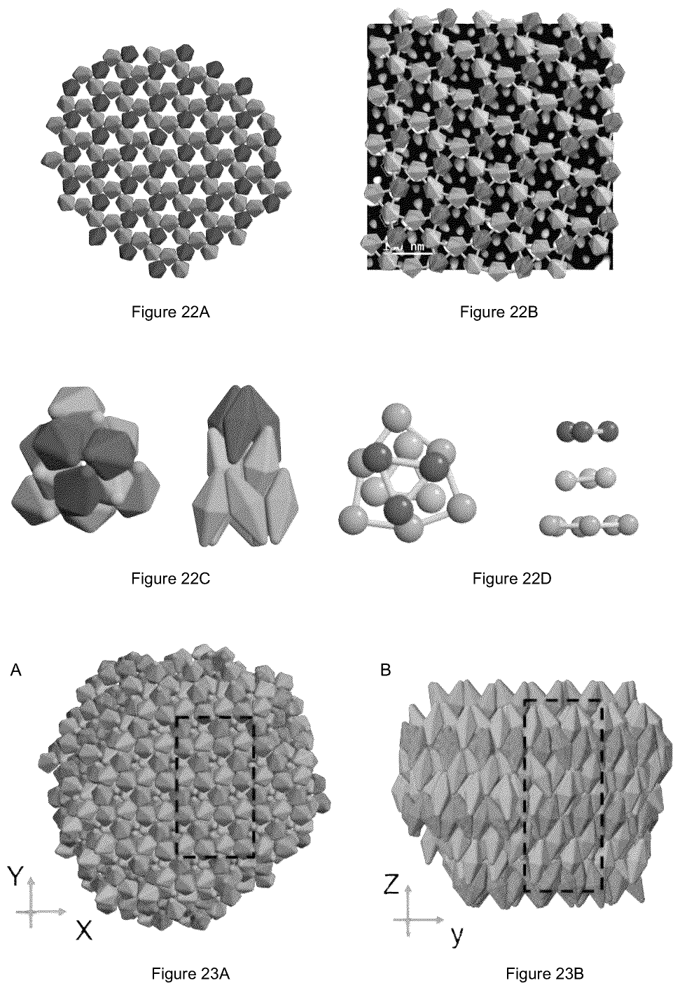

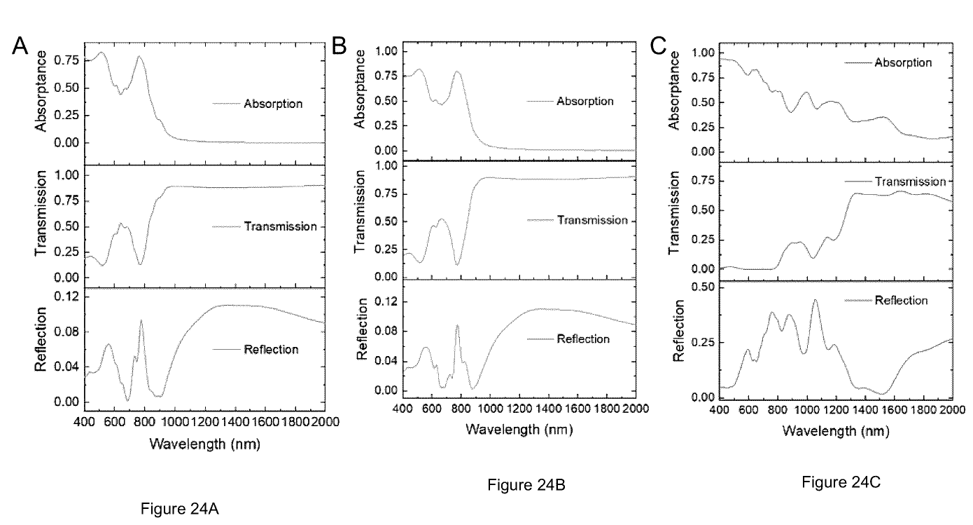

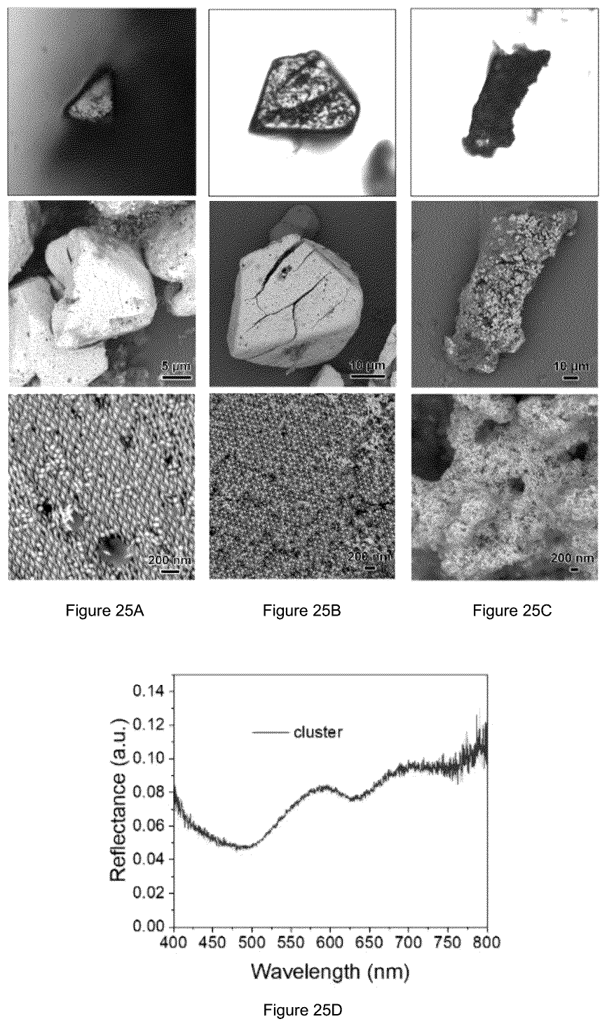

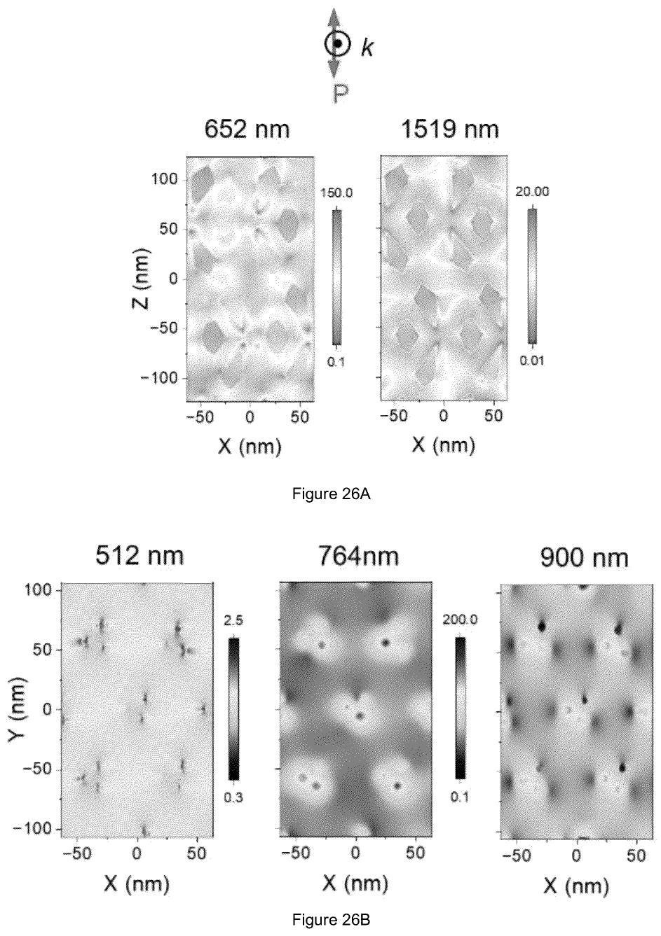

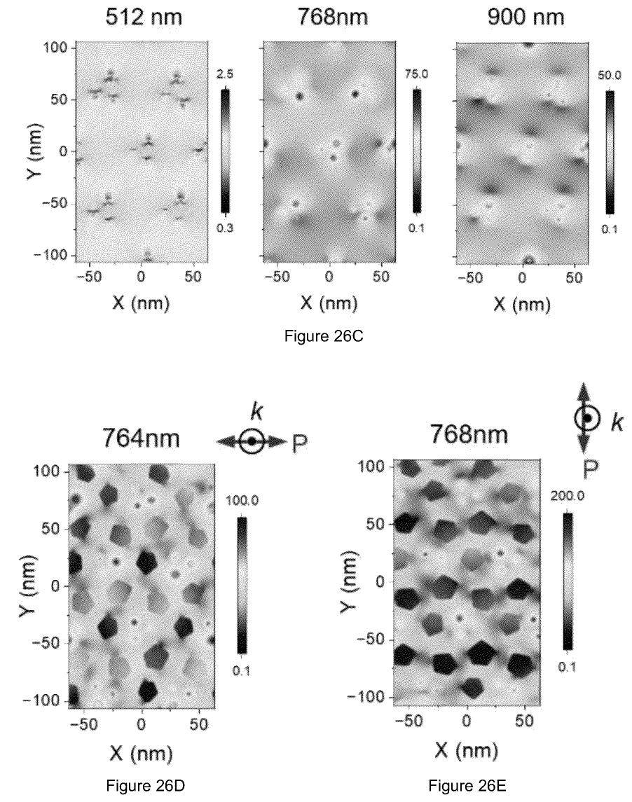

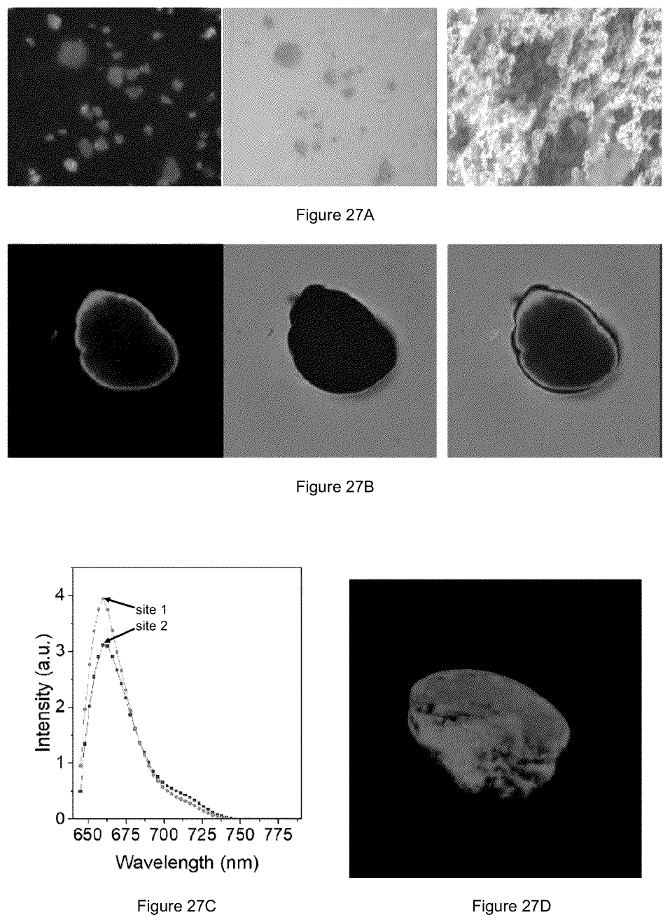

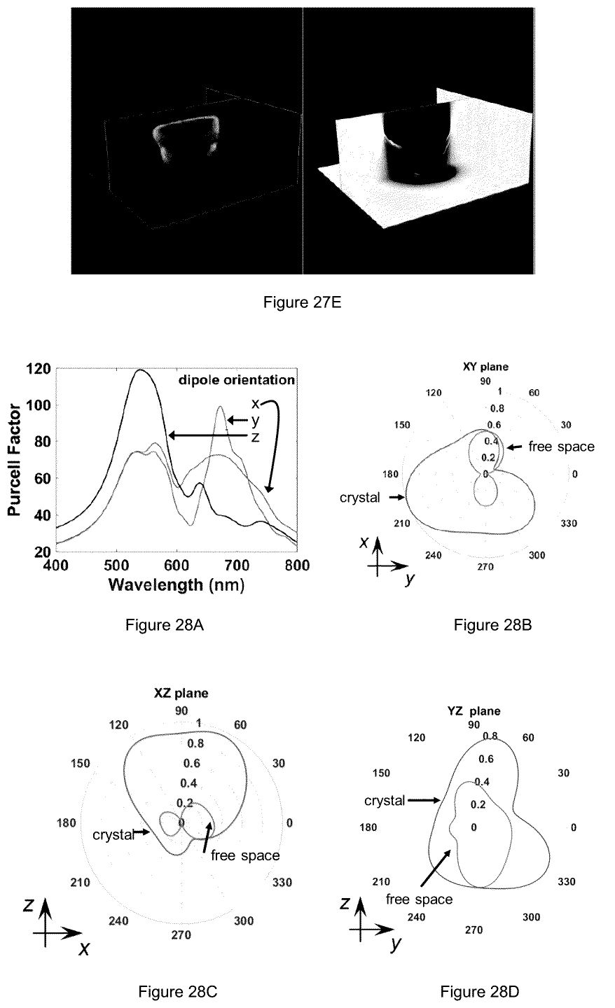

FIG. 11A-11B are schematic simulations of the ripening of kagome superlattices from a mixture of kagome and rhombohedral superlattices. The aspect ratio of the bipyramids is 2.6 and r₀=0.90 (11A) and r₀=0.50 (11B).

FIG. 11C-11D are 3D models of the rhombohedral (C) and kagome (D) lattices.

FIG. 11E-11H are schematics of the 3D superlattices in FIGS. 11A and 11B. (E) and (F) are top and side views of the kagome superlattice, (G) and (H) are the top and side views of the voids and pore topology.

FIG. 12 is a series of Bond-orientational order diagrams (BOOD) of 7-layered kagome superlattice with different cutoff radius as indicated.

FIG. 13 is a schematic of the distorted kagome superlattice obtained from simulation its triangle-shield tiling (lines) constructed by connecting the centers of particles (circles).

FIG. 14A-14D is a series of positional mappings (top panels), bipyramid positions (second panel), bipyramid facet registration relative to kagome lattice (third panel), and schematic illustration of the bipyramid facet registration (bottom layer) in (A) the first layer, (B) second layer, (C) bilayer kagome superlattice, and (D) experimental bilayer kagome superlattices observed in a cross-sectional TEM image.

FIG. 15A is schematic illustration of the possible assembly pathway and bipyramid-bipyramid packing in the kagome superlattice.

FIG. 15B is a schematic illustration of the formation of bipyramid twisting.

FIG. 16 is a schematic illustration of the assembly pathway and the stacking of the kagome superlattices into colloidal crystals.

FIG. 17A-17B are schematics illustrations of (A) regular and (A) distorted kagome superlattices obtained from self-assembly simulations. Hexagons in the regular kagome superlattice are transformed into shields in the distorted kagome superlattice to maximize DNA-hybridization.

FIG. 18A is a schematic of a domain boundary parallel to lattice.

FIG. 18B is a schematic of the sample preparation of cross-sectional TEM analysis.

FIG. 18C is a high magnification HAADF-STEM image of one bipyramid showing the pentagonal crystal domains and domain boundaries.

FIG. 18D is a HAADF-STEM image of the kagome superlattice showing the lattice orientation relative to the domain boundaries of the bipyramids.

FIG. 19A is a series of schematics of stacking of kagome layers into superlattices.

FIG. 19B is a schematic illustration of the kagome packing in the 3D crystals.

FIG. 19C is a cross-sectional SEM image (left panel) and the corresponding schematic illustration of the kagome stacking in a 3D crystal.

FIG. 19D is a schematic illustration of the identical kagome arrays in different layers of the crystals.

FIG. 20 is a series of schematics of the simulated process of crystal assembly and bipyramid twisting, and the orientation and position of bipyramids in a crystal containing nine kagome layers.

FIG. 21 is a series of schematic illustrations of the rhombohedral unit cell in different viewing angles.

FIG. 22A is a kagome lattice formed in simulation with the bipyramids in different orientations.

FIG. 22B is a cross-sectional TEM image overlapped with a simulated kagome array.

FIG. 22C-22D are schematic representations of the bipyramid positional order and orientation in (C) a scheme and (D) a ball-stick diagram.

FIG. 23A-23B are schematic illustrations of the kagome lattices used in optical spectrum simulations with (A) the incident light along the z-axis, perpendicular to the top facet of the kagome superlattice and (B) the incident light along the x-axis, perpendicular to the side facet of the kagome superlattice.

FIG. 24A-24C are simulated absorption, transmission, and reflection spectra of the kagome superlattices under (A) x-polarized, (B) y-polarized, and (C) z-polarized light excitation. The incident light was along the z-axis in (A) and (B) and along the x-axis in (C).

FIG. 25A is a series of Optical microscopy images (top panels), low magnification SEM images (middle), and high-magnification SEM images (bottom panels) of side facets of kagome lattices.

FIG. 25B is a series of Optical microscopy images (top panels), low magnification SEM images (middle), and high-magnification SEM images (bottom panels) of top facets of kagome lattices.

FIG. 25C is a series of Optical microscopy images (top panels), low magnification SEM images (middle), and high-magnification SEM images (bottom panels) of clusters of PAEs.

FIG. 25D is graph showing an experimentally measured reflection spectrum of one large cluster made of bipyramids.

FIG. 26A is a heat map of the electric fields of one crystal in the x-z planes under 652-nm (left panel) and 1519-nm (right panel) light excitation. The light is incident along y-axis and polarized along z-axis.

FIG. 26B is a heat map of the electric fields of the surface of the kagome lattice in the x-y planes under x-polarized light excitation. The light was incident along z-axis and excitation wavelength is labelled in each panel.

FIG. 26C is a heat map of the electric fields of the surface of the kagome lattice in the x-y planes under y-polarized light excitation. The light was incident along z-axis and excitation wavelength is labelled in each panel.

FIG. 26D is a heat map of the electric fields of a cross section of the second kagome lattice in the x-y planes under x-polarized light excitation. The light was incident along z-axis and excitation wavelength is labelled in each panel.

FIG. 26E is a heat map of the electric fields of a cross section of the second kagome lattice in the x-y planes under y-polarized light excitation. The light was incident along z-axis and excitation wavelength is labelled in each panel.

FIG. 27A is a fluorescence optical microscopy image (left panel), bright-field optical microscopy image (middle panel), and SEM image (right panel) of clusters made of Cy5-doped bipyramids.

FIG. 27B is a fluorescence confocal microscopy image (left panel), bright-field confocal microscopy image (middle panel) and merged confocal image (right panel) of clusters made of Cy5-doped bipyramids.

FIG. 27C is a pair of fluorescence spectra of the clusters at different locations.

FIG. 27D is a 3D rendering of the Cy5-doped bipyramid clusters.

FIG. 27E is a cross sectional confocal fluorescence image of Cy5-doped bipyramid clusters.

FIG. 28A is a series of simulated Purcell factor curves of a dipole located inside the crystal and directed along each of the x, y, and z axes.

FIG. 28B-28D are simulated far-field radiation patterns of a random dipole cloud located inside the crystal and same cloud in the free space over (B) the XY plane, (C) the XZ plane, and (D) the YZ plane. All amplitudes are normalized to the same intensity.

### DETAILED DESCRIPTION

Colloidal crystal engineering with DNA allows for the design of diverse superlattices with tunable lattice symmetry, composition, and spacing. Most of these structures follow the complementary contact model (CCM) of maximizing DNA hybridization on building blocks and producing relatively close-packed lattices. However, since non-space-filling shapes with imperfectly aligned particle facets are also possible, the final structures of colloidal crystals can be difficult to predict. Methods of the disclosure provide conditions for preparation of rhombohedral cells with and without distortion by tuning two dimensional parameters of the DNA-modified nanoparticles.

The method according to the disclosure can advantageously be used to assemble low-symmetry kagome superlattices from DNA-modified metal nanoparticle bipyramids engaged only in partial DNA surface matching, rather than close-packed configurations, this allows for alignment variations that can help tune the optical properties of the superlattices. The bipyramid dimensions and DNA length can be engineered for two different superlattices with rhombohedral unit cells, one with unidirectionally oriented bipyramidal nanoparticles and one composed of a periodic stacking of nanoparticles arranged in kagome layers. Enabled by the partial facet alignment, the kagome superlattices exhibit lattice distortion, bipyramid twisting, and planar chirality.

Kagome superlattices of the disclosure can be further conjugated with Cy-5 dyes to serve as cavities with high-density optical states and large Purcell factors along lateral directions, leading to strong dipole radiation along the z axis and facet-dependent light emission. Such complex optical properties can make these materials attractive for lasers, displays, and quantum sensing constructs.

Methods of making colloidal kagome superlattices using programmable atom equivalents (PAEs) in accordance with the disclosure can include cooling the functionalized PAEs in a controlled manner to induce crystal assembly upon hybridization of the sticky ends of oligonucleotides on one PAE with the sticky ends of oligonucleotides of an adjacent PAE. A multi-stage cooling process can be used. For example, the cooling process can include cooling the PAEs from a first temperature to a second temperature at a first cooling rate, cooling the PAEs from the second temperature to a third temperature at a second cooling rate, and cooling the PAEs from the third temperature to a fourth temperature at a third cooling rate. The PAES are metal nanoparticles functionalized with oligonucleotides, each containing an anchor region and a linker region with a self-complementary sticky ends. While reference is made herein to “metal nanoparticles,” it should be understood that the nanoparticles can be single metals or mixtures of metals, such as metal alloys.

In any of the methods disclosed herein, the metal nanoparticles can be prepared by any methods known in the art. For example, a solution of pentagonal nanoparticle seeds of a single metal or metal alloy can be mixed with a solution containing a surfactant and metal ions of the same metal(s) or a different metal(s) than the pentagonal nanoparticle seeds. The metal ions adhere to the seed, growing into metal nanoparticles with a bipyramid shape, the surfactant in the solution, weakly binds to the surface of the metal nanoparticles of bipyramid shape resulting in surfactant stabilized metal nanoparticles. The metal ions can be supplied by any organic or inorganic water-soluble salt (e.g., HAuCl₄); the surfactant can be an ammonium quaternary surfactant, for example, the surfactant can be one or more of cetyltrimethylammonium chloride and cetyltrimethylammonium bromide.

The metal in the metal nanoparticles can be gold, silver, or combinations thereof. The metal in the metal nanoparticles can be the same metal as the metal nanoparticle seeds. The metal in the metal nanoparticles can be different than the metal in the metal nanoparticle seeds.

The PAEs can have a bipyramid shape with an aspect ratio of about 2.5 to about 3.5; for example, about 2.5, 2.6 2.7, 2.73, 2.8, 2.9, 3.0, 3.1, 3.2, 3.5 or any values therebetween. The bipyramid nanoparticles can have a length as shown in FIG. 5B ranging from about 60 nm to about 160 nm; for example, the PAEs can have a length of about 60 nm, 62 nm, 65 nm, 70 nm, 73 nm, 75 nm, 80 nm, 85 nm, 88 nm, 92 nm, 95 nm, 100 nm, 105 nm, 110 nm, 115 nm, 120 nm, 125 nm, 130 nm, 135 nm, 140 nm, 145 nm, 150 nm, 155 nm, 160 nm or any values therebetween.

The surfactant stabilized metal nanoparticles can be functionalized with the oligonucleotides using any known methods. For example, the anchor region can be thiolated for attachment to the nanoparticles. The thiolated oligonucleotides can interact more strongly with the metal nanoparticles than the surfactant, thus displacing the weakly bound surfactant. Thiolated oligonucleotides can be prepared, for example, by reducing DNA anchor strands with dithiothreitol in phosphate buffer and purifying the thiolated DNA anchor strands using a nucleic acid purification desalting column. Other known methods for thiolating an oligonucleotide can be used herein as well. The linker region can have a self-complementary GCGC sticky end, for example.

The oligonucleotides can have a length of about 6 nm to about 20 nm. For example, the oligonucleotides can have a length of about 6 nm, 7 nm, 8 nm, 9 nm, 10 nm, 12 nm, 14 nm, 16 nm, 18 nm, 20 nm or any values therebetween.

The anchor strands can have about 26 to 30 number of bases. For example, the anchor strands can have about 26, 28, or 30 bases or any number of bases therebetween.

The linker strands can have about 20 to 24 number of bases. For example, the linker strands can have about 20, 22, or 24 bases or any number of bases therebetween.

The linker strands can further comprise one or more polyethylene glycol blocks to vary the length of the linker strands.

The linker stands can further include a Cy-5 doped end. For example, the linker can include a Cy-5 linked to a GTA sequence at one end.

In methods of the disclosure, the PAEs are driven into the colloidal crystal arrangement through a multi-stage cooling process, whereby the PAEs arrange by virtue of interaction of hybridization of the linker sticky ends of the oligonucleotide of adjacent PAEs. For example, the colloidal crystal can be formed using three-stage cooling process in which the PAEs are cooled from a first temperature to a fourth temperature in three interval cooling stages using three cooling rates.

During the cooling process, the PAEs are cooled from a first temperature to a second temperature. The first temperature can be about 68° C. to about 72° C. For example, the first temperature can be about 68, 69, 70, 70, 72° C. or any values therebetween. The second temperature can be at least 5° C. lower than the first temperature. For example, the second temperature can be about 5, 6, 7 or 8° C. lower than the first temperature. According to the disclosure, the PAEs can be further cooled from a second temperature to a third temperature, the third temperature can be at least 20° C. lower than the second temperature. For example, the third temperature can be about 20, 21, 22 or 23° C. lower than the second temperature. Even further, the PAEs can be cooled down from a third temperature to a four temperature. The fourth temperature can be at or about room temperature. As used herein, the term room temperature refers to a temperature of about 25° C.

For example, the cooling process can include cooling from a first temperature to a second temperature at a first cooling rate of about 0.1° C./10 min to about 0.2° C./10 min. For example, the first cooling rate can be 0.1° C./10 min, 0.12° C./10 min, 0.15° C./10 min, 0.18° C./10 min, 0.2° C./10 min, or any values therebetween or ranges defined by such values. The process can further include cooling from the second temperature to a third temperature at a second cooling rate of about 0.1° C./20 min to about 0.2° C./20 min. For example, the second cooling rate can be 0.1° C./20 min, 0.12° C./20 min, 0.15° C./20 min, 0.18° C./20 min, 0.2° C./20 min, or any values therebetween or ranges defined by such values. The process can further include cooling from the third temperature to a fourth temperature at a third cooling rate of about 0.1° C./10 min to about 0.2° C./10 min. For example, the third cooling rate can be 0.1° C./10 min, 0.12° C./10 min, 0.15° C./10 min, 0.18° C./10 min, 0.2° C./10 min, or any values therebetween or ranges defined by such values. The first cooling rate can be equal to the third cooling rate. The first cooling rate can be different than the third cooling rate.

Colloidal superlattices of PAEs according to the disclosure can have a rhombohedral unit cell. The PAEs in rhombohedral colloidal superlattices can be metal nanoparticles with bipyramid shape and oligonucleotides attached to the surface. The superlattice can have a complex rhombohedral unit cell comprised of clusters of about 6 PAEs occupying each lattice point (FIG. 3D). The PAEs can tilt within each cluster as illustrated in FIG. 2H due to partial facet alignment, tilting of the PAEs can lead to planar chirality and distortion of the kagome pattern. As used herein the term kagome pattern refers to a uniform 2-dimensional tiling of equilateral triangles and regular hexagons, arranged such that each hexagon is surrounded by triangles and vice versa as illustrated in FIG. 17A. A distorted kagome pattern, can be formed by the tiling of equilateral triangles and hexagonal shields as illustrated in FIG. 17B. As used herein the term kagome layer refers to an array of PAE clusters organized in 2-dimensions in a regular or a distorted kagome pattern.

In embodiments, a colloidal crystal with a rhombohedral unit cell can be formed by the stacking of six distorted kagome layers with alternating chirality along the z-axis. The distorted kagome layers can be offset about ½ along the x direction and about \sqrt{3}/6 along the y axis within one unit cell. In embodiments, colloidal Kagome superlattices can be formed of clusters of bipyramidal PAEs.

Without intending to be bound by theory it is believed that PAEs with bipyramid nanoparticles with low aspect ratios (i.e., 2.5-3.5) and DNA strands with lengths between about 60 nm and 160 nm form distorted kagome superlattices upon following the controlled cooling process of the disclosure. It has been observed that DNA strands of lengths shorter than about 60 nm do not allow for bipyramid tilting leading to unidirectionally oriented bipyramids with no facet alignment and formation of a simple rhombohedral unit cell with three-fold symmetry. Without intending to be bound by theory, tt is believed that strands longer than 160 nm can lead to different type of crystals or simply cluster, and would not form the desired crystals with kagome distortions.

The colloidal kagome superlattice can have complex lattice plasmon resonance modes, which depend on the light incident direction and polarization. These direction-dependent properties can be linked to the shape of the PAEs coupled with the array of the PAEs within the superlattice and the space between PAEs. For example, electric fields can be enhanced around the bipyramid tips, which are perpendicular to the x-y plane except for the slight tilting due to oligonucleotide hybridization. The same enhancement cannot be expected in a different plane since the other planes are dominated by the sides of the bipyramids, rather than the tips.

When conjugated with dyes, kagome superlattices can further exhibit anisotropic light emission where the emitted light depends on the facet of the superlattice. For example, when doped with Cy5 molecular dye, the fluorescence emission of the top facets (perpendicular to the bipyramid tips) can be about six times the emission of the side facets. This contrasts with the emission observed in randomly oriented clusters of bipyramids, where the emission is about the same for all facets. Kagome superlattices of the disclosure can emit light in a range between 650 and 775 nm. The linker strands can be doped with any known dye.

The low symmetry kagome superlattices can also have facet dependent refractive properties, i.e, facets can have different refractive indices, one along z and a different one along the other two directions leading to the structure being birefringent.

Kagome superlattices of the disclosure can serve as cavities with high-density optical states and high emission enhancement factor (Purcell factor). For example, Purcell factors can have a value of at least 25 at a wavelength between 650 and 775 nm. Because of the strong directionality of the bipyramid arrangement, the emission can be enhanced more strongly for in-plane dipoles (along the x and y-axes) than along the out-of-plane z axis, this can result in stronger light emission along the z-direction (perpendicular to the dipole axis).

### Examples

These examples are intended to show the design of low-symmetry colloidal crystals and kagome superlattices and their potential in the engineering of quantum-lattice systems with complex lattice plasmon resonances and facet-dependent light emission properties. The observed structures and properties make these lattices attractive and previously unidentified materials for lasers, displays, and optical sensors. It has been advantageously recognized that PAE can be arranged with partial facet alignment, which expands the structural possible with DNA mediated colloidal crystal assembly. Conventionally, the complementary contact model (CCM) lead to the general expectation of a perfect facet alignment. Thus, the structures of the disclosures and methods of making such structures were not expected to be producible from a PAE mediated strategy.

These examples of leveraging the tunable parameters of DNA length and sequence along with non-space-filling PAEs paved the way to exotic colloidal crystal architectures not attainable with conventional build blocks and with emerging and previously unidentified optical properties.

### Chemical Reagents

Cetyltrimethylammonium chloride (CTAC), cetyltrimethylammonium bromide (CTAB), AgNO₃, ascorbic acid (AA), HAuCl₄, NaCl, sodium citrate, dithiothreitol (DTT), and NaHB₄ were purchased from sigma. Sodium Dodecyl Sulfate (SDS) was purchased from Millipore. Trimethylammonium chloride was purchased from Thermo Scientific. Triethoxysilane was from Acros. Cy3 and Cy5 were purchased from Glen Research. Chemicals were used without additional purification.

### Synthesis of Au Bipyramids

Penta-twinned Au nanoparticles were prepared and used as seeds to initiate the growth of pentagonal bipyramids. A 10 ml solution of HAuCl₄ (0.25 mM), cetyltrimethylammonium chloride (CTAC, 50 mM), and sodium citrate (5 mM) was prepared, additionally a 0.25 ml of NaHB₄ (25 mM) was freshly prepared and added into the HAuCl₄ solution. The solution turned from yellow to brownish, indicating the formation of Au seed particles. After being stirred at room temperature for 30 min, the seed solution was aged in an oil bath at 80° C. for 90 min. The solution color changed from brown to red. The seed solution was stored at room temperature.

The growth of Au bipyramids of varied sizes was realized by controlling the seed solution volume that was added to a growth solution. In a water bath at a temperature of 30° C., the growth solution was prepared by mixing the following solutions under magnetic stirring: 100 ml of CTAB (100 mM), 5 ml of HAuCl₄ (10 mM), 1 ml of AgNO₃ (10 mM), 2 ml of HCl (2 M), and 0.8 ml of AA (0.1 M) in sequence. Au seed solution was then added into the growth solution. After 2 hours in the water bath, the bipyramids were washed to remove excess chemicals and dispersed in 10 ml of water. To remove impurities, 5 ml of bi-pyramid solution was mixed with 2 ml CTAC solution (0.1 M) and 1 ml NaCl solution (2 M), which was thoroughly mixed using Vortex and left without agitation at room temperature overnight. The large bipyramids precipitated driven by depletion force and small impurity nanospheres remained in the supernatant that was carefully removed using pipettes. This process was repeated twice.

### DNA-Functionalization

Au pentagonal bipyramids of aspect ratios ranging from 2.1 to 3.8 (FIGS. 1B, 5, and Table 1) were prepared and functionalized with thiolated oligonucleotides (i.e., thiolated DNA anchor strands).

TABLE 1
Dimensions of Au pentagonal bipyramids
used in colloidal crystal assembly.

| | Length | Diameter | Aspect ratio | Precursor |
|---|---|---|---|---|
| Sample | (l) (nm) | (d) (nm) | (r) | volume/type |
| 1 | 46.6 | 20.7 | 2.3 | 4.5 mL HAuCl₄ |
| 2 | 65 | 23.8 | 2.73 | 10000 μL seeds |
| 3 | 88 | 32 | 2.8 | 500 μL seeds |
| 4 | 103 | 35.5 | 2.87 | 300 μL seeds |
| 5 | 120 | 40 | 2.9 | 200 μL seeds |
| 6 | 135 | 42 | 3.1 | 150 μL seeds |
| 7 | 155 | 48 | 3.2 | 100 μL seeds |
| 8 | 173 | 53 | 3.3 | 50 μL seeds |
| 9 | 325 | 81 | 4.0 | 10 μL seeds |

In a typical procedure, 5 nmol of DNA anchor strands were used for each 1 mL original nanoparticle solution, and 10 mL bipyramid solutions were concentrated in 1 mL for DNA functionalization. The DNA anchor strands (Table 2) were reduced using dithiothreitol (0.1 M in 170 mM phosphate buffer) for 1 hour at room temperature and purified using a nucleic acid purification desalting column. DNA linker strands of different length were prepared by adding PEG blocks to the linker strand sequence in Table 2.

TABLE 2
DNA sequences for bipyramid assembly

| | | |
|---|---|---|
| Thiolated | TCA ACT ATT CCT ACC TAC | SEQ ID |
| Anchor | AAA AAA AAA A - SH | NO: 1 |
| strand | | |
| Linker | GTA GGT AGG AAT AGT TGA | SEQ ID |
| strand | GCGC | NO: 2 |
| Dye-doped | Cy5-GTA GGT AGG AAT AGT | SEQ ID |
| linker | TGA | NO: 3 |
| strand | | |

Meanwhile, the bipyramids were purified by washing against water twice to remove excess CTAB. After the second wash, the supernatant was carefully removed and reduced DNA anchor strands were added to the bipyramids. Afterwards, SDS (0.1%) and (0.1 M) phosphate-buffered saline (PBS) were added in a volume up to a final concentration of 0.01% and 0.01 M, respectively. The mixture was sonicated to fully disperse the bipyramids and shaken overnight at 1000 rpm. Then, NaCl salt solutions (2 M) were added to promote the ligand replacement on bipyramid surfaces. The salt concentration was gradually increased to 0.05, 0.1, 0.2, 0.3, 0.4, and 0.5 M within a 30 min time interval. For 1 mL bipyramid solution, the volume of 2 M NaCl solutions was 25.6, 27.0, 58.5, 65.4, 73.5, and 83.3 mL added each 30 min. After the mixture was shaken for 24 hours, the mixture was centrifugated, and excess DNA anchor strands were removed by washing in 0.01% SDS twice.

The bipyramids were dispersed in 100 μl of a 0.5 M NaCl solution with 0.01% SDS, 0.01 M PBS, with final Au concentration of about 50 mM. Ten nanomole of DNA linker strands of different lengths containing self-complementary GCGC sticky ends (Table 2) were stirred into the bipyramid solution to produce functionalized gold bipyramids to use as programmable atom equivalents (PAEs). Dye doped linker strands were also added to samples intended for investigation of light-emitting properties.

### Colloidal Crystallization Via Controlled Cooling

A solution of PAEs was stabilized at 70° C. in a thermal cycler (Life Technologies) before proceeding with the three-stage cooling process.). The temperature was brought down stepwise, first from 70° C. to 66° C. at a rate of 0.1° C./10 min, the next cooling step was from 66° C. to 45° C. at a rate of 0.1° C./20 min, and the last cooling step from 45° C. to 25° C. was performed at a cooling rate of 0.1° C./10 min. The bipyramids assembled into colloidal crystals with a crystal structure depending on bipyramid dimensions and DNA length (FIG. 6A)

### Microscopy Sample Preparation: Crystal Fixation and Stabilization

A fixing step was performed to prevent the crystal organization form being disrupted by further sample preparation steps needed for microscopy analysis. After assembly, the crystals were transferred into 1.5 mL centrifuge tubes and 0.5 M NaCl solution was added to a final volume of 1 mL. Ten microliter trimethylammonium chloride 50 wt % in methanol was added to the tube, and after 30 min, 5 μL of triethoxysilane were also added. The mixtures were shaken at 900 rpm overnight. Free silica was removed by washing the crystals with water three times. The solid crystals were drop-casted on silicon wafers or TEM grids for electron microscopy characterization.

Thin sections of the crystals were prepared by embedding the crystals in a polymer resin, embed 812 from Electron Microscopy Sciences, and sectioned into 100 nm thin films on TEM grids for electron microscopy characterization.

### Microscopy Characterization of Colloidal Crystals

SEM images of the colloidal crystals were acquired on a JEOL JSM-7900FLV scanning electron microscope under an accelerating voltage of 10 kV and a backscattered electron detection mode. TEM images of bipyramids and sectioned colloidal crystals were acquired in a JEOL ARM2000F transmission electron microscope operating at an accelerating voltage of 200 kV.

Microscopy observations showed that the type of three-dimensional (3D) arrays formed by the PAEs depended on the aspect ratio of the bipyramids and the length of the DNA strand (FIG. 6A). The aspect ratio was defined as the length to diameter ratio (FIG. 5B).

In bipyramids with low aspect ratios, for example 2.3, formation of 3D crystals composed of stacked kagome superlattices was observed (FIG. 1C) with bipyramids adopting different orientations (FIG. 6C). For bipyramids of intermediate aspect ratios (2.8-3.2), the assembled superstructures depended on the length of DNA strands with long DNA strands leading to kagome superlattices and short strands to rhombohedral lattices. A high-magnification scanning electron microscopy (SEM) image showed that the {111}facet has trimers of bipyramids sitting in a triangular lattice, which resulted in the individual bipyramids arranged in a kagome superlattice (FIG. 1D). Within each trimer, the bipyramids have different orientations, as confirmed by SEM in an exposed side facet (FIG. 1E). Bipyramids with high aspect ratios of up to 4 formed rhombohedral crystals (FIG. 6B) with unidirectional bipyramid orientation (FIGS. 1F-1G, 6A, and 7). To investigate the bipyramid positional order in the kagome superlattices, 50 nm thick sections were prepared and imaged using transmission electron microscopy (TEM). TEM images showed up to 45-48 successive layers of kagome superlattices with lattice positions offset laterally from one layer to the next (FIGS. 1H and 11), leading to flower-like stacking motifs (FIG. 1J). This unique assembly contained diverse bipyramid local motifs in different facets of the resulting colloidal crystals (FIG. 1K).

### Establishing Thermodynamic Stability of the Colloidal Superlattices Based on the Complementary Contact Model (CCM)

Molecular dynamics (MD) simulations were performed to ascertain if the kagome superlattices were minimum free-energy structures following the CCM and to better elucidate the observed structures. The DNA modified gold PAEs were modeled as hard cores with surrounding DNA shells (FIGS. 2A and 8), with the DNA modeled as patches on the PAE surface (table 3). The patch—patch distance where DNA hybridization occurs, r₀, represents roughly twice the DNA length. DNA hybridization was modeled by a shifted Gaussian potential (FIG. 8). MD simulations used a discrete element method (DEM) implemented in HOOMD-blue software package. DEM was used to calculate the interaction between anisotropic particles by summing the interactions between all the pairs of geometric features, such as faces, edges, and vertices of the particle shape. The modeled DNA—Pentagonal bipyramids (DNA-PBPs) were represented as a hard pentagonal bipyramid core with 47 attractive patches distributed on the surface of the core. The coordinates of the core vertices and the patches are listed in Table 3.

TABLE 3
Coordinates of the core particle vertices (upper)
and of the patches (lower) in MD simulation model

| | | | | | | | |
|---|---|---|---|---|---|---|---|
| Index | x | Y | z | Index | x | y | z |

Vertices of pentagonal bipyramid

| | | | | | | | |
|---|---|---|---|---|---|---|---|
| 1 | 1.0000 | 0.0000 | 0.0000 | 5 | 0.3090 | −0.9511 | 0.0000 |
| 2 | 0.3090 | 0.9511 | 0.0000 | 6 | 0.0000 | 0.0000 | 2.6000 |
| 3 | −0.8090 | 0.5878 | 0.0000 | 7 | 0.0000 | 0.0000 | −2.6000 |
| 4 | −0.8090 | −0.5878 | 0.0000 | | | | |

Patch coordinates

| | | | | | | | |
|---|---|---|---|---|---|---|---|
| 1 | 0.3090 | −0.9511 | 0.0000 | 25 | 0.2060 | 0.6340 | −0.8667 |
| 2 | 0.4363 | −0.3170 | 0.8667 | 26 | −0.0637 | 0.8300 | 0.0000 |
| 3 | 0.1030 | −0.3170 | 1.7333 | 27 | −0.4363 | 0.7089 | 0.0000 |
| 4 | 0.2060 | −0.6340 | 0.8667 | 28 | −0.8090 | 0.5878 | 0.0000 |
| 5 | 0.4363 | −0.3170 | −0.8667 | 29 | −0.5393 | 0.0000 | 0.8667 |
| 6 | 0.1030 | −0.3170 | −1.7333 | 30 | −0.2697 | 0.1959 | 1.7333 |
| 7 | 0.2060 | −0.6340 | −0.8667 | 31 | −0.5393 | 0.3919 | 0.8667 |
| 8 | 0.5393 | −0.6340 | 0.0000 | 32 | −0.5393 | 0.0000 | −0.8667 |
| 9 | 0.7697 | −0.3170 | 0.0000 | 33 | −0.2697 | 0.1959 | −1.7333 |
| 10 | 1.0000 | 0.0000 | 0.0000 | 34 | −0.5393 | 0.3919 | −0.8667 |
| 11 | 0.4363 | 0.3170 | 0.8667 | 35 | −0.8090 | 0.1959 | 0.0000 |
| 12 | 0.3333 | 0.0000 | 1.7333 | 36 | −0.8090 | −0.1959 | 0.0000 |
| 13 | 0.6667 | 0.0000 | 0.8667 | 37 | −0.8090 | −0.5878 | 0.0000 |
| 14 | 0.4363 | 0.3170 | −0.8667 | 38 | −0.1667 | −0.5129 | 0.8667 |
| 15 | 0.3333 | 0.0000 | −1.7333 | 39 | −0.2697 | −0.1959 | 1.7333 |
| 16 | 0.6667 | 0.0000 | −0.8667 | 40 | −0.5393 | −0.3919 | 0.8667 |
| 17 | 0.7697 | 0.3170 | 0.0000 | 41 | −0.1667 | −0.5129 | −0.8667 |
| 18 | 0.5393 | 0.6340 | 0.0000 | 42 | −0.2697 | −0.1959 | −1.7333 |
| 19 | 0.3090 | 0.9511 | 0.0000 | 43 | −0.5393 | −0.3919 | −0.8667 |
| 20 | −0.1667 | 0.5129 | 0.8667 | 44 | −0.4363 | −0.7089 | 0.0000 |
| 21 | 0.1030 | 0.3170 | 1.7333 | 45 | −0.0637 | −0.8300 | 0.0000 |
| 22 | 0.2060 | 0.6340 | 0.8667 | 46 | 0.0000 | 0.0000 | 2.6000 |
| 23 | −0.1667 | 0.5129 | −0.8667 | 47 | 0.0000 | 0.0000 | −2.6000 |
| 24 | 0.1030 | 0.3170 | −1.7333 | | | | |

DEM was used to calculate the excluded volume interaction between the cores, using the Weeks-Chandler-Anderson (WCA) potential:

$${V}_{WCA} ( r ) = \{ \begin{matrix}4 {ε}_{WCA} [ {( \frac{{σ}_{WCA}}{r} )}^{12} - {( \frac{{σ}_{WCA}}{r} )}^{6} ] - 4 {ε}_{WCA} [ {( \frac{{σ}_{WCA}}{{r}_{cut}} )}^{12} - {( \frac{{σ}_{WCA}}{{r}_{cut}} )}^{6} ] & r < {r}_{cut} \\ 0 & r \geq {r}_{cut}\end{matrix}$$

Where r was the contact distance, the distance between the closest points between pair of geometric features and

$${r}_{c u t} = {2}^{\frac{1}{6}} {σ}_{WCA} .$$

The attractive interaction between the patches was modeled by the shifted Gaussian (SG) potential:

$${V}_{S G} ( r ) = {ε}_{S G} exp exp [ - \frac{1}{2} {( \frac{{r}_{p a t c h} - {r}_{0}}{{σ}_{SG}} )}^{2} ] ,$$

where r_{patch} is the distance between patches, r₀ is the distance where the potential becomes its minimum, ε_{SG} and σ_{SG} are the depth and the width of the potential respectively.

r_{cut}=r₀ was set to match the tail of the WCA potential and the potential minimum of the SG potential, and thus the rounding radius of the hard pentagonal bipyramids was set to be

$$rr = \frac{\frac{{{r}_{0}}^{\frac{1}{6}}}{2}}{2} .$$

All simulations were performed in the NVT ensemble with periodic boundary conditions applied. The timestep was set at dt=0.0025 and the thermostat coupling constant τ=0.01. In all simulations, the units of energy and distance were ε and σ, respectively. For the self-assembly simulation, the potential parameters used were: s_{WCA}=1.00, σ_{WCA}=0.80σ, r_{cut}=0.90σ, ε_{SG}=0.15ε, σ_{SG}=0.30σ, r₀=0.90σ. The system of DNA-PBPs (N=1,000) was initialized as a vapor phase in a cubic box at a particle volume fraction ϕ=0.01, and it was then equilibrated at a temperature k_{B}T=0.80ε (FIG. 9). Due to attractive interaction, the particles started to aggregate into clusters, and later the clusters merged into a single larger cluster (FIG. 2B). Crystal nucleation occurred at the surface of the particle cluster. The system was at that point equilibrated at a slightly raised temperature (k_{B}T=0.85ε) to facilitate the growth of the crystal nuclei throughout the cluster.

INJAVIS software was used for visualization and to conduct structure analysis. The nucleation and growth of the kagome superlattice was identified through visual inspection of the simulation snapshots and calculation of bond-orientational order diagram (BOOD) and fast Fourier transform (FFT) patterns. BOODs were obtained through projection of the bonds connecting particle centers to their neighbors found within a cutoff radius onto a sphere. FFT patterns were calculated by projecting the particle centers onto a plane and applying the Fourier transform.

The stabilities of the two colloidal crystals was examined by modeling a mixed phase of rhombohedral and kagome superlattices that were annealed in the presence of dispersed bipyramids (FIG. 11). The system was prepared initially with randomly dispersed and randomly oriented PAEs and then equilibrated at a temperature below the hybridization temperature (T/Tₘ˜0.9). A pentagonal bipyramid with an intermediate aspect ratio of 2.6 was selected for these simulations in combination with three different DNA lengths (r₀=0.5σ, 0.7σ, or 0.9σ, where the circumcircle radius of the pentagon of the bipyramids is 1σ). The initial rhombohedral (N=150) and kagome (N=150) lattices were obtained from portions of the previously assembled crystals. The two crystallites were placed in the center of the box and surrounded by a “gas” of individual particles (N=700). Three initially identical systems where then equilibrated at a constant temperature, each using a different single DNA length (either r₀=0.5σ, 0.7σ, or 0.9σ). Systems with shorter DNA strands (r₀=0.5σ and 0.7σ) showed a gradual dissolution of the Kagome crystal, while the rhombohedral crystal grew (k_{B}T=0.60ε and k_{B}T=0.75 s) (FIG. 10). With long DNA strands (r₀=0.9σ), a gradual dissolution of the rhombohedral lattice and simultaneous growth of the kagome superlattice were observed (k_{B}T=0.85ε) (FIG. 9).

Mixed phases of the two superlattices constructed using the same DNA length for each superlattice in a bath of dispersed PAEs were calculated to confirm the thermodynamically preferred phases at given conditions. For r₀=0.9σ, the kagome superlattice grew at the expense of the simpler rhombohedral super-lattice; for r₀=0.5σ and r₀=0.7σ, the reverse occurred. For both cases, the thermodynamically stable crystal grew at the dissolution of the unstable phase. On the basis of the 3D models of kagome superlattice (FIG. 11E-11F), the volume fraction of the bi-pyramids and DNA shells was calculated to be 69% in the kagome superlattice. FIGS. 11G and 11H show the void topology with the volume occupied by bipyramids and DNA shells removed. The simulation results provided an insight on the formation mechanism of the lattices and were consistent with the experimental phases (FIG. 6A).

Without intending to be bound by any theory it is believed that when long DNA strands are used as linkers during assembly, the kagome superlattices form as the thermo-dynamically stable structures as the long DNA strands provide more space between particles to allow the different bipyramid orientations, twisting, and lattice distortions needed to form kagome lattices. Conversely, the rhombohedral phase dominates when short DNA strands are used as linkers as the short DNA strands provide compact bipyramid packing, with unidirectional bipyramid alignment.

Distorted Kagome Superlattices and Bipyramid Twisting from Partial Facet Alignment.

The fast Fourier transform (FFT) patterns were calculated for structure analysis based on the centroids of the pentagonal bipyramids in the simulated kagome superlattice (FIGS. 2C and 2D). The FFT pattern of the simulated crystals showed a sixfold rotational symmetry, consistent with a (triangular) hexagonal arrangement of trimer clusters (FIG. 2C and FIG. 12). In the perpendicular direction, the FFT pattern showed a periodic stacking of 2D kagome superlattices (FIG. 2D). Additional peaks in the in-plane FFT indicated the kagome superlattices were distorted. In contrast to regular kagome superlattices (FIG. 17A) described by a tiling of regular triangles and hexagons, the distorted kagome superlattice (FIG. 17B) formed by the DNA-PBPs was tiled with regular triangles and hexagonal shields that were equilateral but not equiangular

$$( \frac{π}{2} and \frac{π}{6} radians )$$

(FIGS. 2E and 13). A regular kagome superlattice was constructed and equilibrated in MD simulations to check for stability. The regular kagome superlattice was spontaneously distorted to the same lattice obtained in the self-assembly simulations previously described, which confirmed that the distorted kagome superlattice is more stable than the regular kagome superlattice.

Further structural analyses of the stable distorted kagome superlattices showed that the polar axis of each bipyramid within a trimer points in one of three directions out of the kagome plane (FIG. 2F). Specifically, the polar axes cycled and twisted unidirectionally within every trimer, leading to the same planar chirality in each layer (FIG. 2F). The three bipyramids within a trimer twist in opposite directions in adjacent layers, forming right-handed and left-handed layers. The same triangle-shield tiling is also present in the experimental kagome superlattices. The positions of Au bipyramids in the different layers were observed in sequential 50 nm-thick ultramicrotome sections imaged in the TEM. Within one kagome superlattice, the arrangement of bipyramids can be described by a triangle-shield tiling (FIG. 2G), which experimentally ascertained the lattice distortion. An alternative trimer orientation was also observed in the TEM images in agreement with the simulation results (FIG. 14).

The local structural motifs were further analyzed to explain the trimer twisting and kagome superlattice distortion. In principle, the construction of a 2D kagome superlattice could be achieved by repeating the trimer motif in a plane with hexagonal symmetry (FIG. 15A). The pentagonal bipyramid geometry, though, would preclude the formation of trimers with perfect facet alignment among all three bipyramids. The geometry does allow for perfect edge-to-edge alignment for the top or bottom halves of the bipyramids, but such alignment does not allow maximum DNA hybridization and is therefore not preferred by the CCM. However, because the DNA is long enough, both the particle geometry and the CCM permit three bipyramids to form a trimer by bipyramid twisting (FIGS. 2H, 15B, 16, and 17), resulting in lattice distortion.

Experimental verification of the lattice distortion observed in the simulations was performed by analysis of sequential TEM cross-sectional images (FIG. 18B) to consider how the crystalline domains within individual bipyramids aligned relative to the lattice orientation. If the lattices were distorted, parallel alignment of any of the five domain boundaries with any of the three lattice directions within a layer would be expected (FIGS. 21 and 18A). Examination of the TEM images of the cross-sections of the kagome superlattices and analysis of the angles (θ) between bipyramid domain boundaries and kagome superlattice directions (FIGS. 18C-18D) was performed to construct the polar plot in FIG. 2J; this showed that 90% of the bipyramid domain boundaries are within ±6° relative to the kagome superlattice direction. Such a near-normal distribution around 0° suggested parallel alignment of one bipyramid domain boundary to one lattice direction, which experimentally verified the distorted lattices.

To investigate 3D superlattice formation, 2D sectional TEM images were analyzed and used to map the relative position of pairs of adjacent kagome layers, lattice distortion was ignored for simplification. The top and bottom layers of the kagome superlattice are shown in FIG. 3A. The position offset along the x and y axes between the adjacent lattices was respectively ½ and \sqrt{3}/6 for one periodicity unit (FIGS. 3B and 19). The first and seventh layers had the same bipyramid positions and orientations as shown in FIGS. 3C and 20, demonstrating six kagome layers in each repeat unit along the z-direction. Such stacking led to a rhombohedral super unit cell with one six-bipyramid cluster occupying each lattice point and containing a total of 48 bipyramids in three different orientations (FIG. 3D). The six-bipyramid cluster was observed in sectional TEM images with side and top views shown in the top and bottom panels in FIG. 3E. Three distinct bonding states were recognized in the electron microscopy images: top parallel bonding, bottom parallel bonding (FIG. 3F), and staggered bonding (FIG. 3G). In the model shown in FIGS. 3H and S17 the positional order and the bonding states of the bipyramids within one rhombohedral unit cell were delineated. Parallel bonding was found between bipyramids within one kagome layer and staggered bonding between bipyramids in two adjacent layers (FIG. 22).

### Anisotropic Light-Emitting Kagome Superlattices

Finite-Difference Time-Domain (FDTD) simulation of the lattice optical properties. Full electromagnetic simulations based on the FDTD method were performed using commercial software ANSYS LUMERICAL. Johnson and Christy's material model was used for Au. The structure was placed on the x-y plane. For the reflection and field distribution simulations, the structure was illuminated by a plane wave source placed under the z-side of the structure with the injection axis parallel to the surface normal (+i). Perfectly Matched Layer (PML) boundary conditions were applied at ±z boundaries, and periodic boundary conditions were applied at ±x and ±y boundaries. For z-polarized simulations, the structure was rotated by 90 degrees around the x axis. The reflected fields were collected by a near-field field and power monitor located below the source. The electric field inside the structure was collected by a 3D box field monitor surrounding the entire structure. For Purcell factor calculations, a dipole was located inside the crystal. For radiation pattern simulations, a dipole cloud (LUMERICAL built-in object) was located inside the crystal, and the crystal was located inside a transmission box formed by 2D field monitors. The same dipole cloud was used for free space simulations. For the decay rate simulations, single dipoles were placed on the surfaces, and the simulations were performed separately for each face and dipole axis (x, y, z). The radiation pattern and radiative/nonradiative decay rates were calculated using built-in analysis groups. The average decay rate was calculated as

$${τ}_{avg} = ln ( - \frac{{e}^{- {τ}_{x}} + {e}^{- {τ}_{y}} + {e}^{- {τ}_{z}}}{3} )$$

where σ_{x,y,z} were the decay rates for x, y, z-oriented dipoles.

Optical Characterization of kagome crystals. Confocal images were taken using a Leica SP8 Confocal optical microscope. The optical reflectance spectra of single colloidal crystals were measured on a Nikon TI Inverted Microscope coupled to an ANDOR Acton Spectrometer with a Newton EMCCD Camera.

The bipyramid anisotropy and low lattice symmetry rendered the kagome superlattices with complex plasmon resonance modes. To analyze the plasmon resonances, reflection spectra were experimentally measured and simulated using a finite-difference time-domain simulation tool (FIG. 23). Reflection was dependent on light incident directions due to reduced lattice symmetry (FIGS. 4A and 24). The crystals for measuring reflection spectra under x- and z-axis incident light are shown in FIGS. 25A and 25B respectively. A large cluster made of bipyramids of the same size as those assembling into colloidal crystals demonstrated much lower reflectance in the visible spectrum due to the random scattering of disordered bipyramids (FIGS. 25C-25D). The plasmon modes were resolved by plotting the electric fields inside the kagome superlattices at different resonance wavelengths. Depending on the light polarization direction relative to the kagome superlattice orientation, longitudinal (FIG. 26A) and transverse modes (FIGS. 26B-26C) were identified, in which the electric fields were strongly enhanced around the bipyramid tips. For light polarization along the z-axis a longitudinal excitation mode at 652 nm and a weak coupling mode at 1519 nm were observed (FIG. 26A). For x-polarized light, lattice plasmon resonances at distinct excitation wavelengths were observed (FIG. 4B). At 512 nm, transverse dipole resonance modes appeared for the kagome superlattices, producing well-aligned dipoles in each bipyramid tip parallel to light polarization. As the wavelength increased to 764 nm, field enhancement was strongly localized around each bipyramid tip, which was also observed inside the 3D superlattice (FIG. 26D and E).

The light-emitting properties of these kagome superlattices was further investigated by conjugating Cy-5 dyes to the DNA linker strands. The facet-resolved fluorescence spectra revealed a sixfold emission enhancement of the top facets compared to the side facets (FIG. 4C), leading to much brighter red-light emission on the top facets (FIGS. 4D and E). However, the cluster made of randomly oriented and positioned bipyramids exhibited similar light emission along different directions (FIG. 27). These two experiments revealed that the facet-dependent, anisotropic light emission is highly correlated with the reduced symmetry and anisotropic lattice plasmon resonance of the kagome superlattices. The driving factors were further studied through simulations of the effective refractive index of the kagome superlattices and emission properties of the dye molecules inside and on the surface of the crystals. The kagome superlattices were found to be birefringent, i.e., possessed two different refractive indices, depending on the orientation (along z and along the other two directions; FIG. 4F). For the dye molecules inside the crystal, the kagome superlattices acted as a cavity, which determined the density and distribution of optical states and resulted in enhancement or diminution of emission (Purcell effect) and contributed to the shapes that defined the radiation pattern. As shown in the simulations (FIGS. 4G and 28A), the Purcell factor, which is the emission enhancement factor of the cavity, was larger for in-plane dipoles (along x and y axes) than out-of-plane (along z axis) ones, leading to stronger light emission along the z direction (perpendicular to the dipole axis). The effects of the kagome superlattice on the radiation pattern of the dye molecules inside the crystal were also simulated and compared with the emission in free space, where the dye molecules were modeled by a cloud of randomly distributed out-of-phase dipoles. As seen from the simulated radiation patterns over the fundamental planes (FIGS. 4H, and 28B-28D), the kagome superlattices emit light toward the top-down direction, which is responsible for the facet-dependent fluorescence observed in the experiments. For the dye molecules on the surfaces, the plasmonic effects on the crystal surface provided competing enhancement and loss channels. Individual contribution of these competing factors was elucidated through simulated radiative and nonradiative decay rates (FIGS. 4I and 4J). The emitters on the top and side faces exhibited similar radiative and distinct nonradiative decay rates. The nonradiative decay rate (FIG. 4J) at excitation and emission wavelengths was larger for the emitters on the top face, indicating that the enhancement expected from strong field localization around the bipyramid tips (FIG. 26) was redeemed by the nonradiative (loss) channels.

Without intending to be bound by theory, it is believed that that the faceted, anisotropic light emission was driven by the crystal structures serving as anisotropic cavities, as opposed to plasmonic field enhancement on the surfaces. Moreover, the low-symmetry kagome superlattices serving as cavities present much higher emission enhancement factors (Purcell factor) along the x- and y-directions, due to a higher density of optical states along the two directions and leading to stronger radiation along the z axis and facet-dependent, anisotropic light emission.

The foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In the case of conflict, the present description, including definitions, will control.

Throughout the specification, where the compounds, compositions, methods, and/or processes are described as including components, steps, or materials, it is contemplated that the compounds, compositions, methods, and/or processes can also comprise, consist essentially of, or consist of any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

### REFERENCES

1. C. A. Mirkin, R. L. Letsinger, R. C. Mucic, J. J. Storhoff, A DNA-based method for rationally assembling nanoparticles into macroscopic materials. Nature 382, 607-609 (1996).
2. G. M. Whitesides, B. Grzybowski, Self-assembly at all scales. Science 295, 2418-2421 (2002).
3. W. B. Rogers, W. M. Shih, V. N. Manoharan, Using DNA to program the self-assembly of colloidal nanoparticles and microparticles. Nat. Rev. Mater. 1, 1-14 (2016).
4. M. A. Boles, M. Engel, D. V. Talapin, Self-assembly of colloidal nanocrystals: From intricate structures to functional materials. Chem. Rev. 116, 11220-11289 (2016).
5. S. Zhou, J. Li, J. Lu, H. Liu, J.-Y. Kim, A. Kim, L. Yao, C. Liu, C. Qian, Z. D. Hood, Chiral assemblies of pinwheel superlattices on substrates. Nature 612, 259-265 (2022).
6. Z. Li, Q. Fan, Z. Ye, C. Wu, Z. Wang, Y. Yin, A magnetic assembly approach to chiral superstructures. Science 380, 1384-1390 (2023).
7. S. C. Glotzer, M. J. Solomon, Anisotropy of building blocks and their assembly into complex structures. Nat. Mater. 6, 557-562 (2007).
8. S. C. Glotzer, Some assembly required. Science 306, 419-420 (2004).
9. R. J. Macfarlane, B. Lee, M. R. Jones, N. Harris, G. C. Schatz, C. A. Mirkin, Nanoparticle superlattice engineering with DNA. Science 334, 204-208 (2011).
10. Z. Li, Q. Fan, Y. Yin, Colloidal self-assembly approaches to smart nanostructured materials. Chem. Rev. 122, 4976-5067 (2021).
11. T. Wang, J. Zhuang, J. Lynch, O. Chen, Z. Wang, X. Wang, D. LaMontagne, H. Wu, Z. Wang, Y. C. Cao, Self-assembled colloidal superparticles from nanorods. Science 338, 358-363 (2012).
12. Z. Li, C. Qian, W. Xu, C. Zhu, Y. Yin, Coupling morphological and magnetic anisotropy for assembling tetragonal colloidal crystals. Sci. Adv. 7, eabh1289 (2021).
13. Kuzyk, R. Schreiber, Z. Fan, G. Pardatscher, E.-M. Roller, A. Högele, F. C. Simmel, A. O. Govorov, T. Liedl, DNA-based self-assembly of chiral plasmonic nanostructures with tailored optical response. Nature 483, 311-314 (2012).
14. Z. Cheng, M. R. Jones, Assembly of planar chiral superlattices from achiral building blocks. Nat. Commun. 13, 4207 (2022).
15. C. Y. Zheng, W. Hadibrata, S. Kim, G. C. Schatz, K. Aydin, C. A. Mirkin, Large-area, highly crystalline DNA-assembled metasurfaces exhibiting widely tunable epsilon-near-zero behavior. ACS Nano 15, 18289-18296 (2021).
16. S. Lee, H. A. Calcaterra, S. Lee, W. Hadibrata, B. Lee, E. Oh, K. Aydin, S. C. Glotzer, C. A. Mirkin, Shape memory in self-adapting colloidal crystals. Nature 610, 674-679 (2022).
17. Y. Li, W. Zhou, I. Tanriover, W. Hadibrata, B. E. Partridge, H. Lin, X. Hu, B. Lee, J. Liu, V. P. Dravid, Open-channel metal particle superlattices. Nature 611, 695-701 (2022).
18. Z. Cai, Z. Li, S. Ravaine, M. He, Y. Song, Y. Yin, H. Zheng, J. Teng, A. Zhang, From colloidal particles to photonic crystals: Advances in self-assembly and their emerging applications. Chem. Soc. Rev. 50, 5898-5951 (2021).
19. G. Rainó, M. A. Becker, M. I. Bodnarchuk, R. F. Mahrt, M. V. Kovalenko, T. Stöferle, Superfluorescence from lead halide perovskite quantum dot superlattices. Nature 563, 671-675 (2018).
20. Cherniukh, G. Rainó, T. Stöferle, M. Burian, A. Travesset, D. Naumenko, H. Amenitsch, R. Erni, R. F. Mahrt, M. I. Bodnarchuk, Perovskite-type superlattices from lead halide perovskite nanocubes. Nature 593, 535-542 (2021).
21. C. Cherqui, M. R. Bourgeois, D. Wang, G. C. Schatz, Plasmonic surface lattice resonances: Theory and computation. Acc. Chem. Res. 52, 2548-2558 (2019).
22. R. Rengarajan, D. Mittleman, C. Rich, V. Colvin, Effect of disorder on the optical properties of colloidal crystals. Phys. Rev. E71, 016615 (2005).
23. Stein, B. E. Wilson, S. G. Rudisill, Design and functionality of colloidal-crystal-templated materials-chemical applications of inverse opals. Chem. Soc. Rev. 42, 2763-2803 (2013).
24. Y. Wang, J. Chen, Y. Zhong, S. Jeong, R. Li, X. Ye, Structural diversity in dimension-controlled assemblies of tetrahedral gold nanocrystals. J. Am. Chem. Soc. 144, 13538-13546 (2022).
25. E. V. Shevchenko, D. V. Talapin, N. A. Kotov, S. O'Brien, C. B. Murray, Structural diversity in binary nanoparticle superlattices. Nature 439, 55-59 (2006).
26. C. R. Laramy, M. N. O'Brien, C. A. Mirkin, Crystal engineering with DNA. Nat. Rev. Mater. 4, 201-224 (2019).
27. M. He, J. P. Gales, É. Ducrot, Z. Gong, G.-R. Yi, S. Sacanna, D. J. Pine, Colloidal diamond. Nature 585, 524-529 (2020).
28. H. Lin, S. Lee, L. Sun, M. Spellings, M. Engel, S. C. Glotzer, C. A. Mirkin, Clathrate colloidal crystals. Science 355, 931-935 (2017).
29. P. Alivisatos, K. P. Johnsson, X. Peng, T. E. Wilson, C. J. Loweth, M. P. Bruchez Jr., P. G. Schultz, Organization of ‘nanocrystal molecules’ using DNA. Nature 382, 609-611 (1996).
30. D. Nykypanchuk, M. M. Maye, D. Van Der Lelie, O. Gang, DNA-guided crystallization of colloidal nanoparticles. Nature 451, 549-552 (2008).
31. Y. Zhang, F. Lu, K. G. Yager, D. Van Der Lelie, O. Gang, A general strategy for the DNA-mediated self-assembly of functional nanoparticles into heterogeneous systems. Nat. Nanotechnol. 8, 865-872 (2013).
32. M. R. Jones, N. C. Seeman, C. A. Mirkin, Programmable materials and the nature of the DNA bond. Science 347, 1260901 (2015).
33. C. A. Mirkin, S. H. Petrosko, Inspired beyond nature: Three decades of spherical nucleic acids and colloidal crystal engineering with DNA. ACS Nano 17, 16291-16307 (2023).
34. C. Zhang, R. J. Macfarlane, K. L. Young, C. H. J. Choi, L. Hao, E. Auyeung, G. Liu, X. Zhou, C. A. Mirkin, A general approach to DNA-programmable atom equivalents. Nat. Mater. 12,
35. 741-746 (2013).
36. D. Yao, Y. Zhang, X. Zhou, X. Sun, X. Liu, J. Zhou, W. Jiang, W. Hua, H. Liang, Catalytic-assembly of programmable atom equivalents. Proc. Natl. Acad. Sci. U.S.A. 120, e2219034120 (2023).
37. P. A. Gabrys, L. Z. Zornberg, R. J. Macfarlane, Programmable atom equivalents: Atomic crystallization as a framework for synthesizing nanoparticle superlattices. Small 15, e1805424 (2019).
38. M. Girard, S. Wang, J. S. Du, A. Das, Z. Huang, V. P. Dravid, B. Lee, C. A. Mirkin, M. Olvera de la Cruz, Particle analogs of electrons in colloidal crystals. Science 364, 1174-1178 (2019).
39. R. J. Macfarlane, M. N. O'Brien, S. H. Petrosko, C. A. Mirkin, Nucleic acid-modified nanostructures as programmable atom equivalents: Forging a new “table of elements.:. Angew. Chem. Int. Ed. 52, 5688-5698 (2013).
40. M. R. Jones, R. J. Macfarlane, B. Lee, J. Zhang, K. L. Young, A. J. Senesi, C. A. Mirkin, DNA-nanoparticle superlattices formed from anisotropic building blocks. Nat. Mater. 9, 913-917 (2010).
41. M. R. Jones, K. L. Kohlstedt, M. N. O'Brien, J. Wu, G. C. Schatz, C. A. Mirkin, Deterministic symmetry breaking of plasmonic nanostructures enabled by DNA-programmable assembly. Nano Lett. 17, 5830-5835 (2017).
42. M. N. O'Brien, M. R. Jones, B. Lee, C. A. Mirkin, Anisotropic nanoparticle complementarity in DNA-mediated co-crystallization. Nat. Mater. 14, 833-839 (2015).
43. C. R. Laramy, H. Lopez-Rios, M. N. O'Brien, M. Girard, R. J. Stawicki, B. Lee, M. O.

De La Cruz, C. A. Mirkin, Controlled symmetry breaking in colloidal crystal engineering with DNA. ACS Nano 13, 1412-1420 (2018).
44. D. Samanta, W. Zhou, S. B. Ebrahimi, S. H. Petrosko, C. A. Mirkin, Programmable matter: The nanoparticle atom and DNA bond. Adv. Mater. 34, e2107875 (2022).
45. Sánchez-Iglesias, N. Winckelmans, T. Altantzis, S. Bals, M. Grzelczak, L. M. Liz-Marzán, High-yield seeded growth of monodisperse pentatwinned gold nanoparticles through thermally induced seed twinning. J. Am. Chem. Soc. 139, 107-110 (2017).
46. Q. Chen, S. C. Bae, S. Granick, Directed self-assembly of a colloidal kagome lattice. Nature 469, 381-384 (2011).
47. S. A. Mallory, A. Cacciuto, Activity-enhanced self-assembly of a colloidal kagome lattice. J. Am. Chem. Soc. 141, 2500-2507 (2019).
48. X. Mao, Q. Chen, S. Granick, Entropy favours open colloidal lattices. Nat. Mater. 12, 217-222 (2013).
49. S.-H. Lim, T. Lee, Y. Oh, T. Narayanan, B. J. Sung, S.-M. Choi, Hierarchically self-assembled hexagonal honeycomb and kagome superlattices of binary 1 D colloids. Nat. Commun. 8,360 (2017).
50. Sun, A. Souslov, X. Mao, T. C. Lubensky, Surface phonons, elastic response, and conformal invariance in twisted kagome lattices. Proc. Natl. Acad. Sci. U.S.A. 109, 12369-12374 (2012).
51. P. B. Johnson, R.-W. Christy, Optical constants of the noble metals. Phys. Rev. B6, 4370-4379 (1972).
52. M. Spellings, R. L. Marson, J. A. Anderson, S. C. Glotzer, GPU accelerated Discrete Element Method (DEM) molecular dynamics for conservative, faceted particle simulations.
53. Comput. Phys. 334, 460-467 (2017).
54. J. A. Anderson, J. Glaser, S. C. Glotzer, HOOMD-blue: A Python package for high-performance molecular dynamics and hard particle Monte Carlo simulations. Comput. Mater. Sci. 173, 109363 (2020).
55. J. Glaser, T. D. Nguyen, J. A. Anderson, P. Lui, F. Spiga, J. A. Millan, D. C. Morse, S. C. Glotzer, Strong scaling of general-purpose molecular dynamics simulations on GPUs. Comput. Phys. Commun. 192, 97-107 (2015); https://github.com/glotzerlab/hoomd-blue.
56. J. D. Weeks, D. Chandler, H. C. Andersen, Role of repulsive forces in determining the equilibrium structure of simple liquids. J. Chem. Phys. 54, 5237-5247 (1971).
57. Engel, iNJAViS—iNteractive JAva ViSualization. Zenodo, 4639570 (2021); https://zenodo.org/record/4639570#.ZG0SPKXMJaY

What is claimed is:

1. A method of making colloidal kagome superlattices using programmable atom equivalents, the method comprising:
preparing programmable atom equivalents, comprising:
admixing a plurality of pentagonal nanoparticle seeds, one or more metal ions, and a surfactant,
ageing the admixture to grow a plurality of surfactant stabilized metal nanoparticles each having a bipyramid shape with surfactant molecules weakly bounded to a surface thereof, wherein each of the pentagonal nanoparticle seeds comprise a metal or metal alloy,
admixing the plurality of surfactant stabilized metal nanoparticles with a plurality of oligonucleotides, each comprising an anchor region and a linker region to thereby functionalize the plurality of surfactant stabilized metal nanoparticles with the plurality of oligonucleotides to thereby produce the programmable atom equivalents, wherein the anchor region of the oligonucleotide attaches to the surface of the metal nanoparticles, and the linker region comprises a self-complementary GCGC sticky end;
and
cooling the programmable atom equivalents to thereby induce crystallization of the programmable atom equivalents, wherein upon cooling adjacent ones of programmable atom equivalents are adapted to arrange in a desired crystalline structure through hybridization of sticky ends of oligonucleotides of one programmable atom equivalent with sticky ends of oligonucleotides of another programmable atom equivalent, the cooling comprising:
cooling the programmable atom equivalents from a first temperature to a second temperature at a first cooling rate.
cooling the programmable atom equivalents from the second temperature to a third temperature at a second cooling rate,
cooling the programmable atom equivalents from the third temperature to a fourth temperature at a third cooling rate, wherein the first temperature is higher than the second temperature, the second temperature is higher than the third temperature, and the fourth temperature is about room temperature.

2. The method of claim 1, wherein the metal nanoparticles are gold nanoparticles, silver nanoparticles, or combinations thereof.

3. The method of claim 1, wherein the metal ions are the same metal as the metal nanoparticle seeds.

4. The method of claim 1, wherein the metal ions are a different metal than the metal nanoparticle seeds.

5. The method of claim 1, wherein the surfactant is an ammonium quaternary surfactant comprising one or more of cetyltrimethylammonium bromide and cetyltrimethylammonium chloride.

6. The method of claim 1, wherein the anchor region is thiolated.

7. The method of claim 1, wherein the programmable atom equivalents each have an aspect ratio of about 2.5 to about 3.5 and a length of about 60 nm to about 160 nm.

8. The method of claim 1, wherein the oligonucleotide is DNA or RNA.

9. The method of claim 1, wherein the oligonucleotide has a length of about 6 nm to about 20 nm.

10. The method of claim 1, wherein the anchor regions have about 26 to about 30 bases and/or the linker strands have about 20 to about 24 bases.

11. The method of claim 1, wherein the linker strands comprise a dye-doped end, optionally a Cy5-dye doped end.

12. The method of claim 1, wherein the first cooling rate is about 0.1° C./10 min to about 0.2° C./10 min and/or the second cooling rate is about 0.1° C./20 min to about 0.2° C./20 min and/or the third cooling rate is 0.1° C./10 min to about 0.2° C./10 min.

13. The method of claim 1, wherein the first cooling rate and the third cooling rate are equal.

14. The method of claim 1, wherein the first temperature is about 68 to 72° C.

15. The method of claim 1, wherein the second temperature is at least 5° C. lower than the first temperature; and/or wherein the third temperature is at least 20° C. lower than the second temperature.

16. A colloidal kagome superlattice of programmable atom equivalents (PAEs), the superlattice comprising a rhombohedral unit cell formed by 3 right-handed and 3-left handed chiral layers of PAEs, wherein:
the PAEs are pentagonal metal bipyramids with oligonucleotide attached to an outer surface thereof,
the layers stack in alternating chirality, wherein one right handed layer is stacked between two left-handed layers,
each layer comprises a plurality of bipyramids organized in trimers that upon hybridization of the oligonucleotides twist to form a 2D distorted kagome pattern characterized by tiling of equilateral triangles and hexagonal shields.

17. The colloidal kagome superlattice of claim 16, wherein a Purcell factor of the superlattice is at least 25 at a wavelength between 650 and 775 nm.

18. The colloidal kagome superlattice of claim 16, wherein the superlattice is birefringent.

19. The colloidal kagome superlattice of claim 20, wherein the superlattice comprising Cy5-doped PAEs, emits light in a wavelength in a range between 650 and 775 nm.

20. The colloidal kagome superlattice of claim 16, wherein the intensity of the light emitted by a plane perpendicular to the length of the programmable atom equivalents is about 6 times higher than the intensity of the light emitted by a plane parallel to the length of the programmable atom equivalents.


SEQUENCE LISTING (3 sequences)

SEQ ID NO: 1 - DNA, 28 bp, synthetic construct
  source 1..28: mol_type = other DNA
  misc_difference 28: note = Thiol
tcaactattcctacctacaaaaaaaaaa

SEQ ID NO: 2 - DNA, 22 bp, synthetic construct
  source 1..22: mol_type = other DNA
gtaggtaggaatagttgagcgc

SEQ ID NO: 3 - DNA, 18 bp, synthetic construct
  source 1..18: mol_type = other DNA
  misc_difference 1: note = Cyanine 5
gtaggtaggaatagttga